(19) United States
(12) Patent Application Publication
(10) Pub. No.: US 2021/0308666 A1
(43) Pub. Date: Oct. 7, 2021

(54) ASSAY PLATES, SEPARATION SHEETS, FILTERS, AND SAMPLE DEPOSITION MARKS

(71) Applicant: Essenlix Corporation, Monmouth Junction, NJ (US)

(72) Inventors: Stephen CHOU, Princeton, NJ (US); Wei DING, Princeton, NJ (US)

(73) Assignee: Essenlix Corporation, Monmouth Junction, NJ (US)

(21) Appl. No.: 17/268,897

(22) PCT Filed: Aug. 23, 2019
(86) PCT No.: PCT/US19/48024

§ 371 Date: Feb. 16, 2021

Related U.S. Application Data
(60) Provisional application No. 62/722,169, filed on Aug. 23, 2018.

(51) Int. Cl. B01L 3/00; G01N 21/76; G01N 21/64; G01N 21/01

(52) U.S. Cl. CPC B01L 3/508; B01L 2300/043; G01N 21/76; G01N 21/6428; G01N 21/01; B01L 2300/0829; B01L 2200/16; B01L 2300/123; B01L 2400/0677; B01L 2300/168; B01L 2300/165; B01L 2200/0689; B01L 2300/0681; B01L 2300/06; B01L 3/523

(57) ABSTRACT

The present invention is related to the field of bio/chemical sampling, sensing, assays and applications. More particularly, one aspect of the present invention is related to bio/chemical assays, including how to separate two plates, how to separate a certain component from a composite liquid sample and obtain a liquid sample free of the component therein, and how to deposit sample and how operate plates for facilitating assaying.

### CROSS-REFERENCE TO RELATED APPLICATIONS

This application is a National Stage entry (§ 371) application of International Application No. PCT/US2019/048024, filed on Aug. 23, 2019, which claims the benefit of priority of U.S. Provisional Patent Application No. 62/722,169, filed on Aug. 23, 2018, the contents of which is relied upon and incorporated herein by reference in its entirety. The entire disclosure of any publication or patent document mentioned herein is incorporated by reference.

### FIELD

The present disclosure is related to the field of bio/chemical sampling, sensing, assays and applications. More particularly, one aspect of the present invention is related to bio/chemical assays, including how to separate two plates, how to separate a certain component from a composite liquid sample and obtain a liquid sample free of the component therein, and how to deposit sample and how operate plates for facilitating assaying.

### BACKGROUND

The present invention relates to how to separate a component from a composite liquid sample, particularly, the devices and methods for separating a component from a composite liquid sample. In some preferred embodiments, one aspect of the present invention particularly relates to the devices and methods for plasma separation. Conventionally, centrifugation is a common technique used to separate a component from a composite liquid sample based on rotor speed and component differences according to their size, shape, density, viscosity of the medium. This method is laborious, requiring sophisticated equipment and professional handling. It is especially unsuitable for small volume of samples. Small samples are increasingly desired in point-of-care settings and personal health management where miniaturized testing equipment is being quickly developed and commercialized. Other existing arts in the field involve the use of microfluidic channels, eliminating the need of large volume of the sample. However, the manufacturing of microfluidic channels is technically challenging and far from cost-effective. Some other arts take advantage of various filter media, mainly composed of porous materials (like filter paper) or glass fibers, in combination with a housing and supporting apparatus. Such a filter method is usually cost-effective and easy to handle, but often requires discharging or transferring of the filtered product for further analysis or processing.

### SUMMARY

One aspect of the present invention provides an assay device having a separation sheet situated between opposed plates of the device, where one of the plates is very thin and is hard to be separated from the other plate to make the plates open. The separation sheet provides one or both of the following functionality: (1) the separation sheet has an extension portion that is not covered by both plates, and the extension portion can facilitate the opening of the two plates; and (2) the separation sheet prevents direct contact of the opposed plates when the plates are in close proximity, so that one reagent coated on one plate will not contact the other plate, when the plates are not being used for assaying.

Another aspect of the present invention provides an assay device having a separation sheet between opposed plates of the device, and at least one of the plates has a layer of a reagent or a coat of a reagent on a portion of the interior surface of the plate.

Yet another aspect of the present invention provides an assay device having a separation sheet between opposed plates of the device, and at least one of the plates has a retention structure for a liquid reagent for storage or holding, such as a well, cavity, channel, via, or like structure.

Still another aspect of the present invention provides an assay device having the abovementioned retention structure for a liquid reagent on one or both of the opposed plates of the device and a sealing sheet attached to the plate having the retention structure for the liquid reagent that seals the liquid reagent in retention structure.

Another aspect of the present invention provides an assay device having the abovementioned retention structure for a liquid reagent on one plate of the device, an impermeable sealing sheet attached to the plate having the retention structure for the liquid reagent, and the other opposed plate is flexible, resilient, or both, and has a plurality of spacer members that provide for physical plate separation and space or cavity formation between the plates for controlling the thickness of a sample layer and a puncture structure that can pierce or rupture the sealing sheet when the plates are compressed.

Still another aspect of the present invention provides an assay device having a first plate, a second plate, a plurality of spacer members attached to the interior surface of each of the plates that provide for physical plate separation and space or cavity formation between the plates, and a compressible porous member situated between the first and second plates and their respective spacer members, wherein the assay device having the compressible porous member can be used to separate a smaller component from a larger component present in a composite liquid sample by, for example, filtration or size exclusion.

In some embodiments, the present invention provides a device for sample analysis facilitated by a separation sheet, comprising: a first plate; a second plate; a hinge; and a separation sheet, wherein: (a) the first plate and the second plate are movable relative to each other into a different configuration, the different configuration includes an initial configuration, an open configuration, or a closed configuration, wherein: (i) each plate comprises an inner surface that has a sample contact area for contacting a sample deposited between the plates, (ii) at least one of the plates has a thickness of 300 um or less, and (iii) one of the plates has, in the initial configuration, all its edges, other than the edge connected to the hinge, inside the edges of the other plate; (b) the hinge is connected to the first plate and the second plate, and the hinge is configured to allow the first plate and the second plate to rotate around the hinge into a different configuration; and (c) the separation sheet has a thickness of 250 um or less; wherein: in the initial configuration, the separation sheet is sandwiched between the two plates and in contact with the two plates; and the separation sheet has an extended portion that is not covered by any of the plates, wherein the extended portion of the separation sheet is configured to facilitate a separation of the two plates; in the open configuration the first plate and the second plate are partially or entirely separated, and the sample is deposited in the sample contact area on one or both of the plates, and the separation sheet is removed from any contact with one or both of the plates; and in the closed configuration at least part of the deposited sample is compressed by the two plates into a thin layer.

In some embodiments, the present invention provides a device for sample analysis facilitated by a separation sheet, comprising: a first plate; a second plate; a hinge; at least one reagent; and a separation sheet, wherein: (a) the first plate and the second plate are movable relative to each other into a different configuration, the different configuration includes an initial configuration, an open configuration, or a closed configuration, wherein: (i) each plate comprises an inner surface that has a sample contact area for contacting a sample deposited between the plates, and (ii) the second plate has a thickness of 300 um or less; (b) the hinge is connected to the first plate and the second plate, and the hinge is configured to allow the first plate and the second plate to rotate around the hinge into a different configuration; (c) the at least one reagent is coated, in the initial configuration, on the sample contact area of at least one of the plates, and (d) the separation sheet has a thickness of 250 um or less; wherein: in the initial configuration, the separation sheet is sandwiched between the two plates and is in contact with the two plates; and the separation sheet is configured to reduce or prevent, in the initial configuration, the at least one regent on one plate to contact the other plate; in the open configuration the first plate and the second plate are partially or entirely separated, and the sample is deposited in the sample contact area on one or both of the plates, and the separation sheet is removed from any contact with one or both of the plates; and in the closed configuration at least part of the deposited sample is compressed by the two plates into a sample layer of highly uniform thickness.

In some embodiments, the device further comprises, in the initial configuration, at least one reagent coated on the sample contact area of at least one of the plates, wherein the separation sheet reduces or prevents, in the initial configuration, the at least one reagent from contact with the other plate.

In some embodiments, the device further comprises, in the initial configuration, at least one reagent coated on the sample contact area of one of the plates and at least one other reagent in the corresponding location of the sample contact area in the other plate, wherein the separation sheet reduces or prevents, in the initial configuration, the at least one reagent from contact with the at least one other reagent on the corresponding location of the sample contact area of the other plate.

In some embodiments, the device further comprises, spacers attached to inner surface of at least one of the plates and in the sample contact area of one or both of the plates so that in the closed configuration the uniform thickness of the layer is confined by the inner surfaces of the plates and is regulated by the plates and the spacers, and the thickness of the layer is from 0.01 to 200 μm.

In some embodiments, the at least one reagent coated on at least one of the plates is coated on both plates.

In some embodiments, the separation sheet preserves the shelf-life of the at least one reagent, preserves the chemical integrity of the at least one reagent, or both.

In some embodiments, the separation sheet is configured to prevent the at least one reagent on one plate from touching the other plate.

In some embodiments, the separation sheet facilitates the physical separation of the first plate and the second plate from in the initial configuration to an open configuration.

In some embodiments, the separation sheet is constructed of a non-porous material selected from a sheet, fiber sheet, a polymer, a polymer coated paper, or a combination thereof.

In some embodiments, the separation sheet is a material selected from polystyrene, PMMA, PC, COC, COP, or a combination thereof.

In some embodiments, the hinge is configured to substantially maintain a dihedral angle of from 0 to 180 degrees between the first plate and the second plate before and after (0 degrees) an external force is applied to the plates.

In some embodiments, in the initial configuration the second plate has all edges, other than one edge connected to the hinge, inside the corresponding edges of the first plate.

In some embodiments, in the initial configuration the second plate has at least one edge, other than one edge connected to the hinge, inside the corresponding edge of the first plate.

In some embodiments, in the initial configuration the separation sheet has at least one edge that extends over the corresponding edge of the first plate and second plate.

In some embodiments, in the initial configuration the separation sheet has at least one edge that extends over the corresponding edge of the first plate and second plate, and the size of the separation sheet is configured to facilitate opening or separation of the first plate and the second plate from the initial configuration having a dihedral angle of about 0 degrees to the open configuration having a dihedral angle greater than about 0 degrees.

In some embodiments, the present invention provides a method for using a device for sample analysis facilitated by a separation sheet, comprising: (i) obtaining a device of any prior claim having a separation sheet; (ii) removing the separation sheet from the space between the first plate and the second plate; (iii) depositing a sample for analysis when the device is in an open configuration; (iv) after (iii), closing the two plates into a closed configuration, wherein at least part of the sample is deposited in the open configuration; and (v) applying a compression force by pressing the two plates together to form a layer of highly uniform thickness confined by the inner surfaces of the plates.

In some embodiments, the method further comprises a step (vi) of analyzing the sample.

In some embodiments, the method further comprises a step (vi) of analyzing the sample by an enzyme-linked immunosorbent assay (ELISA), radioimmunoassay (RIA), immunoblot analysis, immunofluorescent assay (IFA), immunohistochemistry, immunoelectron microscopy (IEM), or immuno-luminescence.

In some embodiments, the sample contains a biomarker selected from a protein, a small molecule, a cell, a particle, a nucleic acid, or a combination thereof.

In some embodiments, the method further comprises a step (v) of removing the compression force and where the hinge maintains a dihedral angle between the first plate and the second plate that is from 1 to 30 degrees, including any intermediate values and ranges, from the dihedral angle before the compression force is removed.

In some embodiments, the first plate and the second plate comprise rectangular planar members.

In some embodiments, the first plate and the second plate comprise transparent planar members.

In some embodiments, the first plate and the second plate include a uniform gap or cavity therebetween when in a closed configuration.

In some embodiments, the sample contact area is disposed on the inner surface of the first plate.

In some embodiments, the sample contact area comprises a predetermined area configured to make contact with a sample and confine the sample to that predetermined area.

In some embodiments, the separation sheet comprises a flexible planar member including a non-porous material configured to prevent liquid, reagents, debris, or a combination thereof from permeating through the separation sheet.

In some embodiments, the separation sheet is removably attachable to the inner surface of at least the first plate or the second plate, and the separation sheet is configured to protectively enclose the sample contact area.

In some embodiments, the separation sheet comprises an adhesive for removably attaching to at least one of the first plate and the second plate.

In some embodiments, the separation sheet comprises a pressure sensitive or pressure activated adhesive.

In some embodiments, the adhesive comprises an elastomeric compound.

In some embodiments, the adhesive comprises an elastomeric compound selected from the group consisting of acrylics, bio-based acrylate, butyl rubber, ethylene-vinyl acetate (EVA), styrene block copolymers (SBC), or a combination thereof.

In some embodiments, the separation sheet is soluble in water, is soluble in water associated with a sample, or soluble in the sample deposited in the device.

In some embodiments, the separation sheet is soluble in water or is soluble in water of the sample in device when activate by changed temperature, light, or a combination thereof.

In some embodiments, the separation sheet is transparent.

In some embodiments, the separation sheet is non-transparent.

In some embodiments, the separation sheet has a hydrophobic surface.

In some embodiments, the separation sheet has a 430 thickness of 5 um, 10 um, 20 um, 30 um, 50 um, 100 um, 200 um, 300 um, 500 um, including intermediate values and ranges.

In some embodiments, the material of the separation sheet is selected from glass, metal, glass microfiber, cellulose acetate, cotton linter, cellulose, polyethylene, paper, wood fiber, recycled newspaper, vegetable matter, recycled cloth, cloth rags, cellulose fibers from plants, trees, wood pulp, rice, water plants, cotton, or a combination thereof.

In some embodiments, the present invention provides a device for assaying a sample with a liquid reagent storage site, comprising: a first plate, a second plate, spacers, and a liquid reagent storage site, wherein: the first and second plates are movable relative to each other into different configurations; one or both plates are flexible; the spacers are fixed to the inner surface of the first plate and have a predetermined uniform height; the liquid reagent storage site is on the first plate, the second plate, or both; wherein: one of the different configurations is an open configuration where the plates are partially or entirely separated apart, the spacing between the plates is not regulated by the spacers, and the sample is deposited on one or both plates; and one of the different configurations is a closed configuration configured after the sample deposition in the open configuration, and in the closed configuration at least part of the deposited sample is compressed by the two plates into a continuous layer.

In some embodiments, the liquid reagent storage site is a plurality of wells on one or both of the plates and any liquid reagent stored in wells is sealed with a film between the first plate and second plate.

In some embodiments, the present invention provides a device for assaying a sample, comprising: a first plate, a second plate, a storage well, a sealing film, and a liquid reagent, wherein: (a) the first plate and the second plate are movable relative to each other into a different configuration, the different configuration includes an initial configuration, an open configuration, or a closed configuration, wherein: (i) the first plate and the second plate, respectively, comprise an inner surface that has a sample contact area for contacting a sample deposited between the first plate and the second plate, and (ii) the first plate having a thickness of 1,000 microns or less, (b) the storage well having a depth of 250 microns less, and is on the inner surface of the second plate, and (c) the sealing film is a flexible sheet having a thickness of 150 um or less, wherein, in the initial configuration, the liquid reagent is substantially in the storage well and the sealing film is configured to cover the opening of the storage well to keep the liquid reagent inside the storage well, wherein: the initial configuration is a configuration in which both plates are in contact with the sealing film; the open configuration is a configuration in which the first plate and the second plate are partially or entirely separated, and the sample is deposited on one or both plates; and the closed configuration is a configuration in which (i) the sealing film is removed from the space between the first plate and the second plate, and (ii) at least part of the sample is compressed by the first plate and the second plate into a layer of highly uniform thickness, and the uniform thickness of the layer confined by the inner surfaces of the plates is in the range of 0.01 to 200 μm.

In some embodiments, the present invention provides a device for assaying a sample, comprising: a first plate, a second plate, a storage well, a sealing film, and a liquid reagent, wherein: (a) the first plate and the second plate are movable relative to each other into a different configuration, the different configuration includes an initial configuration, an open configuration, or a closed configuration, wherein: (i) the first plate and the second plate, respectively, comprise an inner surface that has a sample contact area for contacting a sample deposited between the first plate and the second plate, and (ii) the first plate having a thickness of 1,000 microns or less, (b) the storage well having a depth of 250 microns less, and is on the inner surface of the second plate, and (c) the sealing film is a flexible sheet having a thickness of 150 um or less, wherein, in the initial configuration, the liquid reagent is substantially in the storage well and the sealing film is configured to seal the opening of the storage well to keep the liquid reagent inside the storage well, wherein: the initial configuration is a configuration in which both plates are in contact with the sealing film; the open configuration is a configuration in which the first plate and the second plate are partially or entirely separated, and the sample is deposited on one or both plates; and the closed configuration is a configuration in which (i) at least part of the sample is compressed by the first plate and the second plate into a layer of uniform thickness, and the uniform thickness of the layer confined by the inner surfaces of the plates is in the range of 0.01 to 200 μm, and (ii) the sealing film is broken by the spacers releasing the reagent stored in the well to the sample.

In some embodiments, the sealing film is a separation sheet.

In some embodiments, the present invention provides a method for having a liquid reagent on a device for sample analysis, comprising: (i) obtaining a device of any prior claim having a sealing film; (ii) removing the sealing film from the space between the first plate and the second plate; (iii) depositing a sample for analysis when the device is in an open configuration; (iv) after step (iii), closing the first plate and the second plate into a closed configuration, wherein at least part of the sample deposited in the open configuration is compressed by the first plate and the second plate into a layer of highly uniform thickness, the uniform thickness of the layer confined by the inner surfaces of the first plate and the second plate is from 0.01 to 200 μm, and the liquid reagent contacts the sample.

In some embodiments, the present invention provides a device for assaying a sample, comprising: a flexible first plate having spacers; a second plate having a storage well, a liquid reagent in the storage well, and a sealing film to seal the liquid reagent in the storage well, wherein the spacers provide spacing between the first plate and a second plate to form a sample layer having a uniform thickness when the plates are configured into a closed configuration after a sample is deposited on the inner surface of either of the plates, and the spacers puncture the sealing film to release the liquid reagent from the storage well to contact the sample when the plates are compressed together.

In some embodiments, the present invention provides a device for separating components of a composite sample, comprising: a first collection plate having spacers attached to one surface of the collection plate; and a filter member atop the spacers of the first collection plate, wherein the filter member receives a liquid containing the composite sample, and a compression force applied to the composite sample separates components in the composite sample into the filter member and the first collection plate.

In some embodiments, the compression force is provided by gravity, centrifugation, a human hand, a hydraulic press, a source of compressed air, or a combination thereof.

In some embodiments, the device further comprises a second collection plate having spacers attached to one surface of the second collection plate; and the second collection plate is placed atop the filter member so that the spacers of the second collection plate contact the filter member.

In some embodiments, filter member separates components in the composite sample so that smaller components are collected in the collection plate and larger components are retained in the filter member.

In some embodiments, the present invention provides a method of separating a composite sample in the device of any prior claim having a filter member, comprising: contacting the filter member with a composite sample; and compressing the composite sample on the filter member in the device having at least one collection plate to separate components of the composite sample.

In some embodiments, the present invention provides a device for assaying a composite sample, comprising: a first collection plate having spacers attached to one surface of the collection plate and having a reagent coated on the surface of the collection plate having the spacers; and a filter member situated atop the spacers of the first collection plate, the filter member having an optical structure on the filter member surface contacting the spacers, wherein: the filter member receives a liquid containing the composite sample; a compression force applied to the composite sample separates components in the composite sample into the filter member and the first collection plate; the reagent coat, if present, contacts separated components in the first collection plate to form a product between the reagent and an analyte; and the optical structure enhances the detection of the product between the reagent and an analyte.

In some embodiments, the device further comprising an optical structure in place of the reagent coat on the first collection plate.

In some embodiments, the optical structure is selected from an optical plate, an optical coat, or a combination thereof.

In some embodiments, the compression force is provided by gravity, centrifugation, a human hand, a hydraulic press, a source of compressed air, or a combination thereof.

In some embodiments, the present invention provides a device for sample analysis facilitated by a mark, comprising: a first plate, a second plate, and a deposition mark, wherein: the first plate and second plate are movable relative to each other into different configurations, including an open configuration and a closed configuration, each of the plates comprises an inner surface that has a sample contact area for contacting a sample to be analyzed, and either or both of the plates has the deposition mark that indicates an approximate location on the plate for depositing the sample, wherein in the open configuration the plates are partially or entirely separated apart, and the average spacing between the sample contact areas of the plates is larger than 300 um; wherein in the closed configuration the first and second plates are compressed together to sandwich the sample into a thin layer of a thickness of 200 μm or less; and wherein the depostion mark is configured to facilitate the analysis of the sample when the plates are in the closed configuration.

In some embodiments, the present invention provides a device for sample analysis facilitated by a mark, comprising: a first plate, a second plate, and a compression mark, wherein: the first plate and second plate are movable relative to each other into different configurations, including an open configuration and a closed configuration, each of the plates comprises an inner surface that has a sample contact area for contacting a sample, and either or both of the plates has the compression mark that indicates an approximate location for iniating a compression of the plates into a closed configuration, wherein in the open configuration the plates are partially or entirely separated apart, and the average spacing between the sample contact areas of the plates is larger than 300 um, and wherein in the closed configuration the first and second plates are compressed together to sandwich the sample into a thin layer of a thickness of 200 μm or less; and wherein the compression mark is configured to facilitate the analysis of the sample when the plates in the closed configuration.

In some embodiments, the present invention provides a device for sample analysis facilitated by a mark, comprising: a first plate, a second plate, and a filling mark, wherein: the first plate and second plate are movable relative to each other into different configurations, including an open configuration and a closed configuration; each of the plates comprises an inner surface that has a sample contact area for contacting a sample; and either or both of the plates has the filling mark that indicates an approximate area that the sample must fill when the plates are in the closed configuration; wherein in the open configuration the plates are partially or entirely separated apart, and the average spacing between the sample contact areas of the plates is larger than 300 um; wherein in the closed configuration the first and second plates are compressed together to sandwich the sample into a thin layer of a thickness of 200 μm or less; and wherein the filling mark is configured to facilitate the analysis of the sample when the plates in the closed configuration.

In some embodiments, the present invention provides a method for assaying a composite sample, comprising: (i) obtaining a device of any prior claim having a first collection plate having spacers attached to one surface of the collection plate and having a reagent coated on the surface of the collection plate having the spacers; and a filter member situated atop the spacers of the first collection plate, the filter member having an optical structure on the filter member surface contacting the spacers, (ii) depositing a sample for analysis on the filter member; and (iii) providing a compression force to the sample on the filter member to separate the composite sample into the filter member and the first collection plate, wherein the reagent coat, if present, contacts separated components in the first collection plate to form a product between the reagent and an analyte; and the optical structure, if present, enhances the detection of the product between the reagent and an analyte.

In some embodiments, the present invention provides a method for sample analysis facilitated by a deposition mark, comprising: (i) obtaining a device of any prior claim having a deposition mark, wherein: the first plate and second plate are movable relative to each other into different configurations, including an open configuration and a closed configuration, each of the plates comprises an inner surface that has a sample contact area for contacting a sample to be analyzed, and either or both of the plates has the deposition mark that indicates an approximate location on the plate for depositing the sample; (ii) depositing the sample on the deposition mark; and (iii) compressing the plates, wherein in the open configuration the plates are partially or entirely separated apart, and the average spacing between the sample contact areas of the plates is larger than 300 um; wherein in the closed configuration the first and second plates are compressed together to sandwich the sample into a thin layer of a thickness of 200 μm or less; and wherein the depostion mark is configured to facilitate the analysis of the sample when the plates are in the closed configuration.

In some embodiments, the present invention provides a method for sample analysis facilitated by a compression mark, comprising: (i) obtaining a device of any prior claim having a compression mark, wherein: the first plate and second plate are movable relative to each other into different configurations, including an open configuration and a closed configuration, each of the plates comprises an inner surface that has a sample contact area for contacting a sample to be analyzed, and either or both of the plates has the compression mark that indicates an approximate location on the plate for compression of the sample; (ii) depositing the sample on the sample contact area; and (iii) compressing the plates on the compression mark, wherein in the open configuration the plates are partially or entirely separated apart, and the average spacing between the sample contact areas of the plates is larger than 300 um; wherein in the closed configuration the first and second plates are compressed together to sandwich the sample into a thin layer of a thickness of 200 μm or less; and wherein the compression mark is configured to facilitate the analysis of the sample when the plates are in the closed configuration.

In some embodiments, the present invention provides a method for sample analysis facilitated by a fill mark, comprising: obtaining a device of any prior claim having a fill mark, wherein: the first plate and second plate are movable relative to each other into different configurations, including an open configuration and a closed configuration, each of the plates comprises an inner surface that has a sample contact area for contacting a sample to be analyzed, and either or both of the plates has the fill mark that indicates an approximate location and amount on the plate for depositing the sample; depositing the sample on the sample contact area to apporimate the fill mark; and compressing the plates, wherein in the open configuration the plates are partially or entirely separated apart, and the average spacing between the sample contact areas of the plates is larger than 300 um; wherein in the closed configuration the first and second plates are compressed together to sandwich the sample into a thin layer of a thickness of 200 μm or less; and wherein the fill mark is configured to facilitate the analysis of the sample when the plates are in the closed configuration.

In some embodiments, the deposition mark or the compression marker comprising a shape of a cross, star, a small circle, or a combination thereof.

In some embodiments, the filling mark comprising a shape of a circle, a square, a triangle, a polygon, or a combination thereof.

### BRIEF DESCRIPTION OF THE DRAWINGS

The drawings are not intended to limit the scope of the present teachings in any way. The drawings may or may not be to scale. The drawings illustrate one or more embodiments of one aspect of the present invention.

FIG. 1 shows a schematic view of the assaying device with the plate separation sheet. Panel (a) shows a perspective view of the assaying device in an open configuration. Panel (b) shows a perspective view of the assaying device in a closed configuration. Panel (c) shows a cross-sectional view of the assaying device of panel (b).

FIG. 2 shows schematics of liquid reagent storage in QMAX card.

FIG. 3 shows schematically exemplary embodiments of the device and method for separating a component from a composite liquid sample.

FIG. 4 is a flow chart for an exemplar method for analyte separation from a liquid separation.

FIG. 5 shows representative images of the filtered products that resulted from different experimental configurations of the device when used for blood plasma separation.

FIG. 6 shows results of a triglyceride (TG) assay using the filtered products from the experimental filtering device as the assay sample and a QMAX device as the assay device.

FIG. 7 shows a schematic of filtering, assay, and read device examples.

FIG. 8 shows an exemplar of an imaging-based imperfection removal and reference method.

FIG. 9 shows a schematic of a device for sample analysis using compressed open flow (COF) in an open configuration (optional hinge connecting the opposing plates not shown).

FIG. 10 shows a schematic of a device for sample analysis using compressed open flow (COF). Panel (a) shows a side view of the COF device in a closed configuration with a camera. Panel (b) shows a top view of the COF device in panel (a) showing an interior sample contact area (dotted line) and a deposition mark (“+”) on the exterior plate surface. Panel (c) shows a top view of a COF device with a deposition mark (“+”) on a first plate positioned directly beneath a camera. Panel (d) shows a top view of the COF device of panel (c) with a compression mark.

### DETAILED DESCRIPTION

The following detailed description illustrates some embodiments of the invention by way of example and not by way of limitation. The section headings and any subtitles used herein are for organizational purposes only and are not to be construed as limiting the subject matter described in any way. The contents under a section heading and/or subtitle are not limited to the section heading and/or subtitle, but apply to the entire description of the present invention.

In some embodiments, the “deposition mark” can be used as a “compression mark”, and vice versa.

A. Assaying Device with Separation Sheet and Liquid Storage

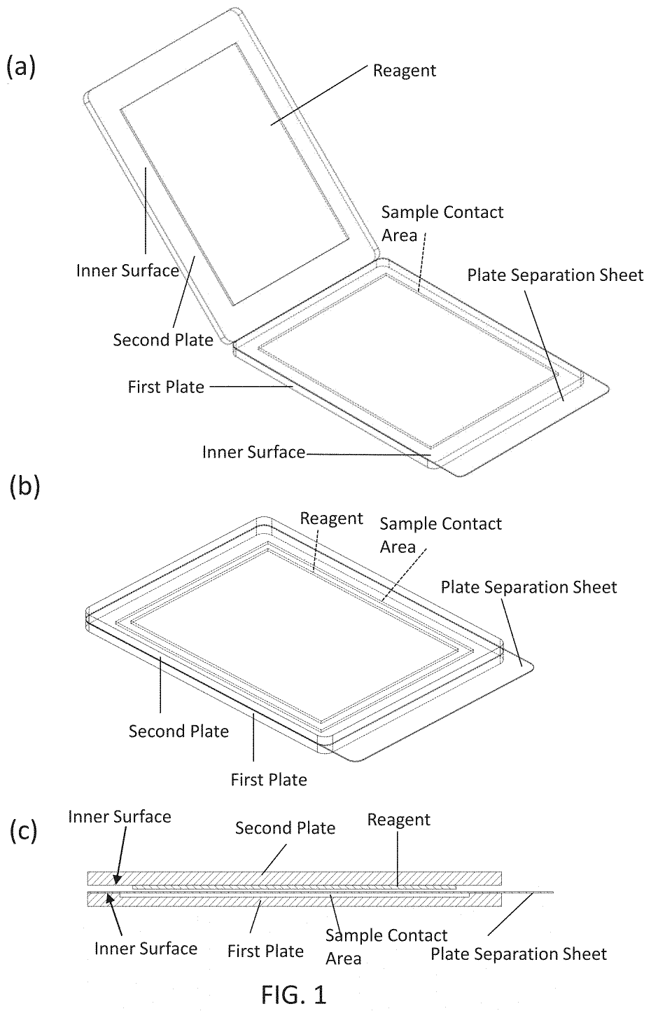

Referring now to FIG. 1, there are shown perspective views of the assaying device that comprises two plates movable to each other in an open configuration and closed configuration, respectively. One aspect of the present invention provides an assaying device comprising a plate separation sheet configured to prevent contamination of a sample contact area of the assaying device and to facilitate the opening thereof. The assaying device includes a first plate, a second plate, a sample contact area, and a plate separation sheet. The first plate and the second plate are configured to move relative to each other between an open configuration and a closed configuration. In the open configuration, the first plate and the second plate are either partially or completely separated from each other. In the closed configuration, the first plate and the second plate are not separated from each other or are compressed together. In the closed configuration, the first plate and the second plate include a small gap there between. In one embodiment, the gap is uniform. In another embodiment, the gap is non-uniform.

The first plate and the second plate include an inner surface, an outer surface, and a perimeter edge. In one embodiment, the first plate and the second plate can be pivotally connected at a portion along their respective perimeter edges. In another embodiment, the first plate and the second plate are hingedly connected via a hinge that interconnects a portion of their respective perimeter edges. In yet another embodiment, a perimeter edge of the second plate is hingedly connected to an inner surface of the first plate. In an alternative embodiment, the first plate and the second plate are hingedly connected via a living hinge. In alternative embodiments, the first plate and the second plate are not connected, rather they are unattached components of the assaying device.

The sample contact area is configured to receive and contain a sample thereon. The sample contact area is disposed on an inner surface of at least one of the first plate or the second plate. In one embodiment, the sample contact area is disposed on the inner surface of the first plate, as shown by FIG. 1. In another embodiment, the sample contact area is disposed on the inner surface of the second plate. In alternative embodiments, a sample contact area is disposed on each of the inner surfaces of the first plate and the second plate. In one embodiment, the sample contact area comprises a recessed portion, as shown by FIG. 1, including a predetermined area configured to receive and confine a sample deposited thereon within the sample contact area when the assaying device is in the closed configuration.

In one embodiment, the assaying device includes one or more spacers configured to regulate the gap formed between the first plate and the second plate when in a closed configuration. The one or more spacers include a height and an inter-spacer distance configured to regulate the gap formed between the first plate and the second plate, when the first plate and the second plate are in the closed configuration. In one embodiment, the one or more spacers each include a uniform height and a constant uniform inter-spacer distance. In another embodiment, the one or more spacers include a non-uniform vertical height and non-constant uniform inter-spacer distance. In one embodiment, the one or more spacers are disposed on the inner surface of the first plate. In another embodiment, the one or more spacers are disposed on the inner surface of the second plate. In yet another embodiment, the one or more spacers are disposed on both of the inner surfaces of the first plate and the second plate. In one embodiment, the one or more spacers are disposed on the sample contact area. In one embodiment, the one or more spacers protrude outwardly relative to the inner surface on which they are disposed. In another embodiment, the one or more spacers protrude perpendicularly outwardly relative to the inner surface on which they are disposed. In another embodiment, the one or more spacers comprise an upstanding cylindrical body including a planar upper surface that defines a pillar shape. In operation, the one or more spacers control the uniformity of a sample deposited within the sample contact area by regulating the diffusion of the sample throughout the sample contact area.

In one embodiment, a reagent can be disposed on the inner surface of the second plate of the assaying device, as shown by panel (a) of FIG. 1. The reagent is configured to mix with a sample after deposition of the sample onto the sample contact area to create a desired reaction for aiding in the detection of a target biomarker within the sample.

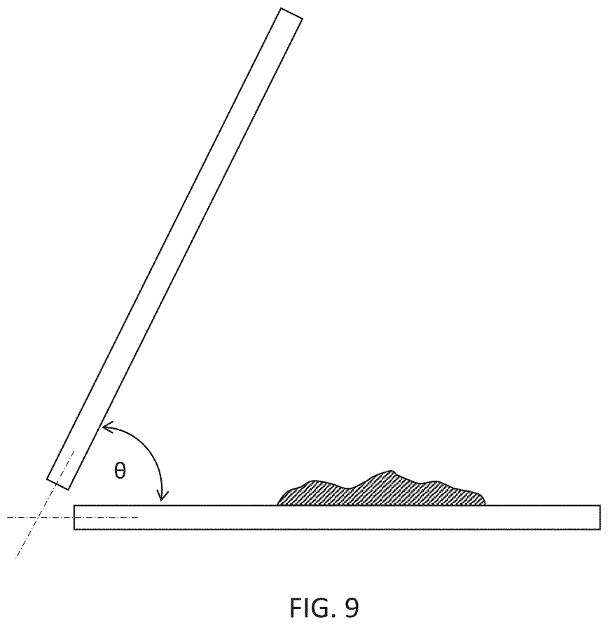

The plate separation sheet can be configured to protect the sample contact area by preventing the contamination thereof by debris, reagents, and/or other contaminants before the deposition of a sample thereon. The plate separation sheet is disposed between the first plate and the second plate so as to be positioned between the first plate and the second plate when in a closed configuration (i.e., referring to FIG. 9, the dihedral angle theta (θ) is greater than 0; in panel (b) of FIG. 1 θ is about 0 degrees), as shown by panel (b) of FIG. 1. In one embodiment, the plate separation sheet comprises a flexible planar membrane, or member, that is removably attachable to the inner surface of first plate. When attached to the inner surface of the first plate, the plate separation sheet encloses the sample contact area thereunder, as shown by FIG. 1. In this way, the sample contact area is protected from debris, and a user can easily remove the plate separation sheet from the first plate to access the sample contact area when employing the assaying device. In one embodiment, the plate separation sheet includes an adhesive material (not shown) for removably attaching the plate separation sheet to the inner surface of the first plate, such as a pressure sensitive or pressure activated adhesive, i.e., elastomeric compounds, including acrylics, bio-based acrylate, butyl rubber, ethylene-vinyl acetate (EVA), styrene block copolymers (SBC), as shown by panel (a) of FIG. 1. In another embodiment, the plate separation sheet comprises an impermeable membrane configured to prevent liquid and debris from permeating therethrough onto the sample contact area and prevent contamination thereof. In yet another embodiment, the plate separation sheet comprises a non-porous membrane configured to prevent liquid and debris from permeating therethrough and onto the sample contact area.

In one embodiment, the plate separation sheet can be removably attachable to the inner surface of the second plate, thereby enclosing the reagent thereunder. In this way, the plate separation sheet protects the sample contact area of the first plate by preventing the reagent from transferring to the sample contact area prior to deposition of a sample thereon. After deposition of a sample onto the sample contact area, the plate separation sheet can be removed from the second plate, thereby enabling the reagent to mix with a sample deposited on the sample contact area when closing the assaying device into the closed configuration.

In one embodiment, the plate separation sheet is configured to facilitate the opening of the assaying device, or the separation of the first plate and the second plate relative to each other into the open configuration. The plate separation sheet protrudes outwardly relative to a perimeter edge of the assaying device when in the closed configuration. In the depicted embodiment, the plate separation sheet extends past a front perimeter edge of the first plate, thereby protruding outwardly from the first plate when in an open and/or closed configuration. When the assaying device is in a closed configuration, the plate separation sheet protrudes outwardly from the first plate and the second plate of the assaying device. In this way, the plate separation sheet provides a graspable portion which a user can grab and lift to separate the first and the second plates from each other and open the assaying device. In another embodiment, the plate separation sheet extends outwardly past all perimeter edges of either the first plate and second plate, with the exception of the edges of the first plate and the second plate that are hingedly connected to each other. In this way, the plate separation sheet protrudes outwardly from more than one edge of the assaying device, thereby providing a larger graspable portion in which a user can grasp to open the assaying device.

The term “plate separation sheet” and “separate sheet” are interchangeable.

A1. A device for sample analysis, comprising:

a first plate, a second plate, a separation sheet, and a hinge, wherein:
(a) the first plate and the second plate are movable relative to each other into a different configuration, said different configuration including an initial configuration, an open configuration, or a closed configuration, wherein:
   (i) each plate respectively comprises an inner surface that has a sample contact area for contacting a sample deposited between the plates, and
   (ii) the second plate has a thickness of 300 um or less,
(b) the hinge connecting the first plate and the second plate, said hinge is configured to allow the first plate and the second plate to rotate around the hinge into a different configuration, and
(c) the separation sheet having a thickness of 250 um or less, wherein the separation sheet is, in the initial configuration, placed between the first plate and the second plate,
wherein the initial configuration is a configuration in which the first plate and the second plate are in contact with the separation sheet,
wherein the open configuration is a configuration in which the first plate and the second plate are partially or entirely separated, and the sample is deposited on one or both of the plates,
wherein the closed configuration is a configuration in which (i) the separation sheet is removed from a space between the two plates, and (ii) at least part of the sample is compressed by the two plates into a layer of highly uniform thickness, the uniform thickness of the layer confined by the inner surfaces of the plates and in the range of 0.01 to 200 μm.
   A2-1. The device of any prior embodiments, further comprising spacers on the sample contact area of one or both plates, wherein, in the closed configuration, the uniform thickness of the layer is confined by the inner surfaces of the plates and is regulated by the plates and the spacers, and is in the range of 0.01 to 200 μm.
   A2-2. The device of any prior embodiments, wherein the hinge is configured to be angle self-maintaining, wherein the hinge substantially maintains an angle, after an external force is removed from the plates that moves the plates from an initial angle (dihedral angle theta about 0 degrees) into another angle (dihedral angle theta greater than 0 degrees).
   A3-1. The device of any prior embodiments, wherein, in the initial configuration, the second plate has all edges, which are not the edge connected to the hinge, inside the corresponding edge of the first plate.
   A3-2. The device of any prior embodiments, wherein, in the initial configuration, the second plate has at least one edge, which is not the edge connected to the hinge, inside the corresponding edge of the first plate.
   A4-1. The device of any prior embodiments, wherein, in the initial configuration, the separation sheet has at least one edge extended over the corresponding edge of the first plate and second plate.
   A4-2. The device of any prior embodiment, wherein, in the initial configuration, the separation sheet has at least one edge extended over the corresponding edge of the first plate and second plate, wherein the size of the separation sheet is configured to facilitate opening between the first plate and the second plate from the initial configuration to the opening configuration.
   A5-1. The device of any prior embodiments, further comprises at least one reagent coated on the inner surface of one of the plates, wherein, the initial configuration, the separate sheet is configured to prevent the at least one reagent on one plate from touching the other plate.
   A5-2. The device of any prior embodiments, further comprises at least one reagent coated on each of the inner surface of the plates, wherein, the initial configuration, the separate sheet is configured to prevent the at least one reagent on one plate from touching the reagent coated on the other plate.
   AM1. A method for using a device for sample analysis, comprising:

(i) obtaining a device of any prior embodiments;

(ii) removing the separation sheet from the space between the first plate and the second plate;

(iii) depositing a sample for analysis when the device is in an open configuration;

(iv) after (iii), closing the two plates into a closed configuration, wherein at least part of the sample deposited in the open configuration is compressed by the two plates into a layer of highly uniform thickness, the uniform thickness of the layer confined by the inner surfaces of the plates and in the range of 0.01 to 200 μm.

AM2-1. The method of any prior embodiments, further comprises a step of analyzing the sample.
AM2-2. The method of any prior embodiments, wherein the analyzing step is performed by an enzyme-linked immunosorbent assay (ELISA), radioimmunoassay (RIA), immunoblot analysis, immunofluorescent assay (IFA), immunohistochemistry, immunoelectron microscopy (IEM), or immunoluminescence.
AM3. The method of any prior embodiments, wherein the analyzing step measures a biomarker in the sample, wherein the biomarker is a protein, small molecule, cell, particle, nucleic acid, or a combination thereof.
Ax. The device of embodiment A2-2, wherein after the external force is removed, the hinge maintains an angle between the first plate and the second plate that is within 5 degrees from the angle before the external force is removed.
A4. The device of embodiment A2-2, wherein after the external force is removed, the hinge maintains an angle between the first plate and the second plate that is within 10 degrees from the angle before the external force is removed.
A5. The device of embodiment A2-2, wherein after the external force is removed, the hinge maintains an angle between the first plate and the second plate that is within 20 degrees from the angle before the external force is removed.
A6. The device of embodiment A2-2, wherein after the external force is removed, the hinge maintains an angle between the first plate and the second plate that is within 30 degrees from the angle before the external force is removed.

### Exemplary Embodiments

1) An assaying device, comprising:

a QMAX card including a first plate and a second plate;

the first plate and the second plate configured to move relative to each other between an open configuration and a closed configuration;

a sample contact area disposed on an inner surface of at least one of the first plate and the second plate, the sample contact area configured to receive a sample thereon;

a plate separation sheet disposed between the first plate and the second plate, the plate separation sheet configured to protect the sample contact area of the assaying device from contamination; and

wherein the plate separation sheet is configured to facilitate the separation of the first plate and the second plate into the open configuration.

2) An assaying device, comprising:

a QMAX card including a first plate and a second plate;

the first plate and the second plate configured to move relative to each other between an open configuration and a closed configuration;

a sample contact area disposed on an inner surface of the first plate, the sample contact area configured to receive a sample thereon;

a plate separation sheet disposed between the first plate and the second plate, the plate separation sheet configured to protect the sample contact area of the assaying device from contamination;

wherein the plate separation sheet protrudes outwardly relative to a perimeter edge of the assaying device when in the closed configuration; and

wherein the plate separation sheet is configured to facilitate the separation of the first plate and the second plate into the open configuration.

3) An assaying device, comprising:

a first plate and a second plate configured to move relative to each other between an open configuration and a closed configuration;

a sample contact area disposed on an inner surface of the first plate, the sample contact area configured to receive a sample thereon;

a plate separation sheet disposed between the first plate and the second plate, the plate separation sheet configured to protect the sample contact area of the assaying device from contamination;

wherein the plate separation sheet protrudes outwardly relative to a perimeter edge of the assaying device when in the closed configuration; and

wherein the plate separation sheet is configured to facilitate the separation of the first plate and the second plate into the open configuration.

4) An assaying device, comprising:

a first plate and a second plate configured to move relative to each other between an open configuration and a closed configuration;

a sample contact area disposed on an inner surface of the first plate, the sample contact area configured to receive a sample thereon;

a reagent disposed on the inner surface of the second plate, the reagent configured to react with a sample disposed onto the sample contact area in the closed configuration;

a plate separation sheet disposed between the first plate and the second plate, the plate separation sheet configured to protect the sample contact area of the assaying device from contamination by the reagent before a sample is deposited on the sample contact area;

wherein the plate separation sheet protrudes outwardly relative to a perimeter edge of the assaying device when in the closed configuration; and

wherein the plate separation sheet is configured to facilitate the separation of the first plate and the second plate into the open configuration.

5) The assaying device of any prior embodiment, further comprising one or more spacers disposed on the inner surface of either the first plate or the second plate or both the first plate and the second plate.
6) The assaying device of any prior embodiment, wherein the one or more spacers are configured to regulate the thickness of the calibration material disposed on either the first plate or the second plate, when the first plate and the second plate are in the closed configuration.
7) The assaying device of any prior embodiment, wherein the one or more spacers protrude perpendicularly outwardly relative to the inner surface on which they are disposed.
8) The assaying device of any prior embodiment, wherein the one or more spacers include a height and an inter-spacer distance configured to regulate an area of a space formed between the first plate and the second plate, when the first plate and the second plate are in the closed configuration.
9) The assaying device of any prior embodiment, wherein the one or more spacers include a uniform height and a constant uniform inter-spacer distance.
10) The assaying device of any prior embodiment, wherein the first plate and the second plate are pivotally connected.
11) The assaying device of any prior embodiment, wherein the first plate and the second plate comprise rectangular planar members.
12) The assaying device of any prior embodiment, wherein the first plate and the second plate comprise transparent planar members.
13) The assaying device of any prior embodiment, wherein the first plate and the second plate include a uniform gap or cavity therebetween when in a closed configuration.
14) The assaying device of any prior embodiment, wherein the sample contact area is disposed on the inner surface of the first plate.
15) The assaying device of any prior embodiment, wherein the sample contact area comprises a predetermined area configured to make contact with a sample and confine the sample to that predetermined area.
16) The assaying device of any prior embodiment, wherein the plate separation sheet comprises a flexible planar member including a non-porous material configured to prevent liquid and debris from permeating there through.
17) The assaying device of any prior embodiment, wherein the plate separation sheet comprises a flexible planar member including an impermeable membrane configured to prevent liquid and debris from transferring therethrough.
18) The assaying device of any prior embodiment, wherein the plate separation sheet is removably attachable to the inner surface of at least the first plate or the second plate, the plate separation sheet configured to enclose the sample contact area thereunder.
19) The assaying device of any prior embodiment, wherein the plate separation sheet is removably attachable to the inner surface of the first plate.
20) The assaying device of any prior embodiment, wherein the plate separation sheet is removably attachable to the inner surface of the second plate and configured to enclose the reagent thereunder.
21) The assaying device of any prior embodiment, wherein the plate separation sheet comprises an adhesive for removably attaching the plate separation sheet to the first plate or the second plate.
22) The assaying device of any prior embodiment, wherein the plate separation sheet comprises a pressure sensitive or pressure activated adhesive.
23) The assaying device of any prior embodiment, wherein the adhesive comprises an elastomeric compound.
24) The assaying device of any prior embodiment, wherein the adhesive comprises an elastomeric compound selected from the group consisting of acrylics, bio-based acrylate, butyl rubber, ethylene-vinyl acetate (EVA), and styrene block copolymers (SBC).
25) The assaying device of any prior embodiment, wherein the separation sheet is soluble in water or soluble in the sample in device.
26) The assaying device of any prior embodiment, wherein the separation sheet is soluble in water or the sample in device when activate by temperate, light, and others.
27) The assaying device of any prior embodiment, wherein the separation sheet is transparent.
28) The assaying device of any prior embodiment, wherein the separation sheet is non-transparent.
29) The assaying device of any prior embodiment, wherein the separation sheet is part of the first plate or the second plate.
30) The assaying device of any prior embodiment, wherein the separation sheet is part of the first plate or the second plate, and is foldable.
31) The assaying device of any prior embodiment, wherein the plate separation sheet protrudes outwardly relative to a perimeter edge at least one of the first plate and the second plate, such that the plate separation sheet protrudes outwardly from the assaying device when in the closed configuration.
32) The assaying device of any prior embodiment, wherein the plate separation sheet protrudes outwardly from a front perimeter edge of the first plate.
33) The assaying device of any prior embodiment, wherein the plate separation sheet extends outwardly past all perimeter edges of either the first plate or second plate, except the edges of the first plate and the second plate that are pivotally connected to each other, for example, with a hinge.
34) The devices or methods of any prior methods, wherein the separation sheet has a hydrophobic surface.
35) The devices or methods of any prior methods, wherein the separation sheet has a thickness of 5 um, 10 um, 20 um, 30 um, 50 um, 100 um, 200 um, 300 um, 500 um, or within a range of any two of these values.
36) The devices or methods of any prior methods, wherein the materials of the separation sheet are selected from polystyrene, PMMA, PC, COC, COP, or another plastic.
37) The devices or methods of any prior methods, wherein the materials of the separation sheet are selected from wood fiber, recycled newspaper, some vegetable matter, recycled cloth, cloth rags, cellulose fibers from plants, trees, wood pulp, rice, water plants, cotton, clothes and others.
38) The devices or methods of any prior methods, wherein the materials of the separation sheet are selected from glass, metal as aluminum and others.
39) The devices or methods of any prior methods, wherein the materials of the separation sheet are selected from glass microfiber, cellulose acetate, cotton linter, cellulose, polyethylene, paper, and others.

It is submitted that one aspect of the present invention has been shown and described in what is considered to be the most practical and preferred embodiments. It is recognized, however, that departures can be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

In some embodiments, the device can store liquid reagent on at least one of the plates.

In some embodiments, one aspect of the present invention provides a device for assaying a sample, comprising:

a first plate, a second plate, spacers, and liquid reagent storage sites, wherein:
i. the first and second plates are movable relative to each other into different configurations;
ii. one or both plates are flexible;
iii. the spacers are fixed to the inner surface of the first plate and have a predetermined uniform height;
iv. the liquid reagent storage sites are on the first plate or second plate or both;

wherein on of the configuration is an open configuration, in which the two plates are partially or entirely separated apart, the spacing between the plates is not regulated by the spacers, and the sample is deposited on one or both plates; and

wherein one of the configurations is a closed configuration, which is configured after the sample deposition in the open configuration, and in the closed configuration at least part of the deposited sample is compressed by the two plates into a continuous layer.

The devices or methods of any prior methods, wherein the device further comprises a liquid reagent stored on one or both plates.

The devices or methods of any prior methods, wherein the device further comprises a liquid reagent stored in wells on one of the plates.

The devices or methods of any prior methods, wherein the device further comprises a liquid reagent stored in wells on both plates.

The devices or methods of any prior methods, wherein the device further comprises a liquid reagent stored in wells on one of the plates and sealed with a film between the first plate and second plate.

The devices or methods of any prior methods, wherein the device further comprises liquid reagent stored in wells on both plates and each sealed with one film between the first plate and second plate.

The devices or methods of any prior methods, wherein the wells on the plate is a single well.

The devices or methods of any prior methods, wherein the wells on the plate is a well array.

The devices or methods of any prior methods, wherein the wells on the plate has a depth of 1 um, 2 um, 5 um, 10 um, 15 um, 20 um, 50 um, 100 um, or within a range of any two of these values.

The devices or methods of any prior methods, wherein the wells on plate has a depth of 100 um, 200 um, 300 um, 500 um, 700 um, 1000 mm, or within a range of any two of these values.

The devices or methods of any prior methods, wherein the wells on plate has an average lateral dimension of 1 um, 5 um, 10 um, 20 um, 50 um, 100 um, or within a range of any two of these values.

The devices or methods of any prior methods, wherein the wells on plate has an average lateral dimension of 100 um, 200 um, 500 um, 800 um, 1000 um, or within a range of any two of these values.

The devices or methods of any prior methods, wherein the wells on plate has an average lateral dimension of 1 mm, 2 mm, 5 mm, 8 mm, 10 mm, 20 mm, or within a range of any two of these values.

The devices or methods of any prior methods, wherein the ratio of total area of wells on plate to store liquid reagent to total area of plate is 1% or less, 2% or less, 5% or less, 10% or less, 15% or less, 20% or less, 30% or less, 40% or less, 50% or less, 60% or less, 70% or less, 80% or less, 90% or less, 95% or less, 99% or less, or within a range of any two of these values.

The liquid reagent(s) stored in wells is one type of the reagent.

The liquid reagents stored in wells can be, for example, several types of the reagents in different wells.

The devices or methods of any prior methods, wherein the plates with stored liquid reagent has a hydrophilic surface.

The devices or methods of any prior methods, wherein the plates without stored liquid reagent has a hydrophobic surface.

The devices or methods of any prior methods, wherein the sealing film for storing a liquid reagent has hydrophobic surface.

The devices or methods of any prior methods, wherein the sealing film has a thickness of 5 um, 10 um, 20 um, 30 um, 50 um, 100 um, 200 um, 300 um, 500 um, or within a range of any two of these values.

The devices or methods of any prior methods, wherein the materials of the sealing film can be, for example, selected from polystyrene, PMMA, PC, COC, COP, or another plastic.

The coating method of liquid reagents on plates can be, for example, by droplet printing, ink jet printing, by air jet printing, or by transfer printing from a stamp.

### B. Liquid Store on a Pair Plates

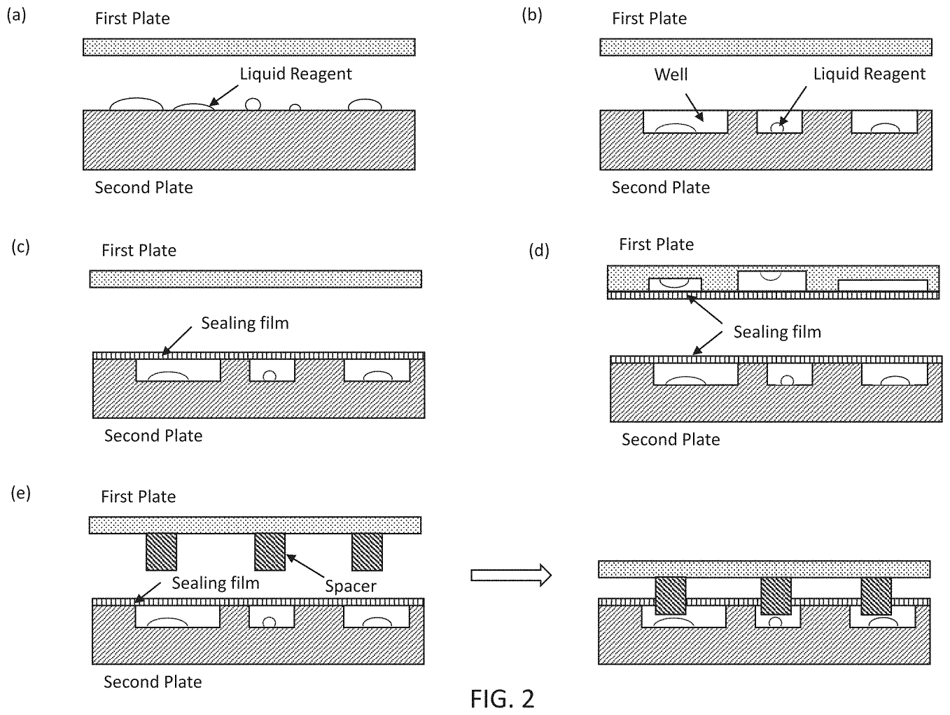

FIG. 2 shows schematics in panels (a) to (e) of liquid reagent storage in, for example, a QMAX card. (a) The device comprises a liquid reagent coated on one of the plates. (b) The device comprises a liquid reagent stored in wells on one of the plates. (c) The device comprises a liquid reagent stored in wells on one of the plates and sealed with a film between the first plate and second plate. (d) The device comprises liquid reagents stored in wells on both plates and each sealed with one film between the first plate and second plate. (e) The device comprises a liquid reagent stored in wells on a second plate and sealed with one film, and the second plate having the sealed wells is opposed by a flexible first plate having spacers attached to the opposing interior surface where the spacers provide spacing between the plates and the spacers can puncture the sealing film to release the reagent. In some embodiments in case (e), in an assaying, a sample is sandwiched between the first and second plate to form a thin layer in the closed configuration; then a further compressing the two plate, the seal is broken by the spacer, releasing the reagent stored in the well to the sample.

In some embodiments, the second plate is flexible. In some embodiments, both the first plate and the second plate are flexible

B1. A device for assaying a sample, comprising:

a first plate, a second plate, a storage well, a sealing film, and a liquid reagent, wherein:
(a) the first plate and the second plate are movable relative to each other into a different configuration, said different configuration includes an initial configuration, an open configuration, or a closed configuration, wherein:
   (i) the first plate and the second plate, respectively, comprise an inner surface that has a sample contact area for contacting a sample deposited between the first plate and the second plate, and
   (ii) the first plate having a thickness of 1,000 um (micron) or less,
(b) the storage well having a depth 250 urn or less, and is on the inner surface of the second plate, and
(c) the sealing film is a flexible sheet having a thickness of 500 urn or less, wherein, in the initial configuration, the liquid reagent is substantially in the storage well and the sealing film is configured to cover the opening of the storage well to keep the liquid reagent inside the storage well,
wherein:
the initial configuration is a configuration in which both plates are in contact with the sealing film;
the open configuration is a configuration in which the first plate and the second plate are partially or entirely separated, and the sample is deposited on one or both plates; and
the closed configuration is a configuration in which (i) the sealing film is removed from the space between the first plate and the second plate, and (ii) at least part of the sample is compressed by the first plate and the second plate into a layer of highly uniform thickness, the uniform thickness of the layer confined by the inner surfaces of the plates and in the range of 0.01 to 200 μm.
   B2-1. The device of any prior embodiments, further comprises a plurality of spacers on the sample contact area of one or both plates, wherein, in the closed configuration, the uniform thickness of the layer is confined by the inner surfaces of the first plate and the second plate and is regulated by the first plate and the second plate and the plurality of spacers, and is in the range of 0.01 to 200 μm.
   B2-2. The device of any prior embodiments, further comprises at least two storage wells on the second plate.
   B2-3. The device of any prior embodiments, wherein said first plate comprises a storage well.
   B2-3. The device of any prior embodiments, further comprising at least two liquid reagents.
   B3-1. The device of any prior embodiments, wherein the sealing film is placed on a plate in the initial configuration is separable from the plate.
   B3-2. The device of any prior embodiments, wherein the sealing film is a separation sheet.
   B4-1. The device of any prior embodiments, wherein, in the initial configuration, the sealing film has at least one edge extended over the corresponding edge of the first plate and the second plate.
   B4-2. The device of any prior embodiments, wherein, in the initial configuration, the sealing film has at least one edge extended over the corresponding edge of the first plate and the second plate, wherein the size of the sealing film is configured to facilitate opening between the first plate and the second plate from the initial configuration to the opening configuration.
   B5-1. The device of any prior embodiments, further comprises at least one reagent coated on the inner surface of one of the plates, wherein in the initial configuration the sealing film is configured to prevent the at least one reagent on one plate from touching the other plate.
   B5-2. The device of any prior embodiments, further comprises at least one reagent coated on each of the inner surface of the plates, wherein in the initial configuration, the sealing film is configured to prevent the at least one reagent on one plate from touching the reagent coated on the other plate.
   B6-1. The device of any prior embodiments, further comprising a hinge that connects the first plate and the second plate, and is configured to allow the two plates to rotate around the hinge into different configurations.
   B6-2. The device of any prior embodiments, wherein the hinge is configured to be angle self-maintaining, wherein the hinge substantially maintains an angle, after an external force that moves the plates from an initial angle into the angle is removed from the plates.
   B7. The device of any prior embodiments, wherein one or both plates are flexible.
   B8. The device of any prior embodiments, wherein the spacers are fixed to the inner surface of the first plate and have a predetermined uniform height.
   B9. The device of any prior embodiment, wherein the spacers are fixed to the inner surface of the first plate, have a flat top, and have a predetermined uniform height.
   BM1. A method for having a liquid reagent on a device for sample analysis, comprising:

(i) obtaining a device of any prior embodiments;

(ii) removing the sealing film from the space between the first plate and the second plate;

(iii) depositing a sample for analysis when the device is in an open configuration;

(iv) after step (iii), closing the first plate and the second plate into a closed configuration, wherein at least part of the sample deposited in the open configuration is compressed by the first plate and the second plate into a layer of highly uniform thickness, the uniform thickness of the layer confined by the inner surfaces of the first plate and the second plate and in the range of 0.01 to 200 μm, and the liquid reagent contacts the sample.

BM2-1. The method of any prior embodiments, further comprising a step: (v) analyzing the sample.
BM2-2. The method of any prior embodiments, wherein the analyzing step is performed using an enzyme-linked immunosorbent assay (ELISA), radioimmunoassay (RIA), immunoblot analysis, immunofluorescent assay (IFA), immunohistochemistry, immunoelectron microscopy (IEM), and immunoilluminescence.
BM3. The method of any prior embodiments, wherein the analyzing step measures a biomarker in the sample, wherein the biomarker is a protein, small molecule, cell, particle, nucleic acid, or a combination hereof.
Bx. The device of any prior embodiments, wherein after the external force is removed, the hinge maintains an angle between the two plates that is within 5 degrees from the angle just before the external force is removed.
A4. The device of any prior embodiments, wherein after the external force is removed, the hinge maintains an angle between the two plates that is within 10 degrees from the angle just before the external force is removed.
A5. The device of embodiment A2-2, wherein after the external force is removed, the hinge maintains an angle between the two plates that is within 20 degrees from the angle just before the external force is removed.
A6. The device of embodiment A2-2, wherein after the external force is removed, the hinge maintains an angle between the two plates that is within 30 degrees from the angle just before the external force is removed.
A7. The devices or methods of any prior methods, wherein the device further comprises a liquid reagent stored on one or both plates.
A8. The devices or methods of any prior methods, wherein the device further comprises a liquid reagent stored in wells on one of the plates.
A9. The devices or methods of any prior methods, wherein the device further comprises a liquid reagent stored in wells on both plates.
A10. The devices or methods of any prior methods, wherein the device further comprises a liquid reagent stored in wells on one of the plates and sealed with a film between the first plate and second plate.
A11. The devices or methods of any prior methods, wherein the device further comprises liquid reagent stored in wells on both plates and each sealed with one film between the first plate and second plate.
A12. The devices or methods of any prior methods, wherein the wells on plate to store liquid reagent is a single well.
A13. The devices or methods of any prior methods, wherein the wells on plate to store liquid reagent is a well array.
A14. The devices or methods of any prior methods, wherein the wells on plate to store liquid reagent has a depth of 1 um, 2 um, 5 um, 10 um, 15 um, 20 um, 50 um, 100 um, or within a range of any two of these values.
A15. The devices or methods of any prior methods, wherein the wells on plate to store liquid reagent has a depth of 100 um, 200 um, 300 um, 500 um, 700 um, 1000 mm, or within a range of any two of these values.
A16. The devices or methods of any prior methods, wherein the well on plate to store liquid reagent has an average lateral dimension of 1 um, 5 um, 10 um, 20 um, 50 um, 100 um, or within a range of any two of these values.
A17. The devices or methods of any prior methods, wherein the well on plate to store liquid reagent has an average lateral dimension of 100 um, 200 um, 500 um, 800 um, 1000 um, or within a range of any two of these values.
A18. The devices or methods of any prior methods, wherein the well on plate to store liquid reagent has an average lateral dimension of 1 mm, 2 mm, 5 mm, 8 mm, 10 mm, 20 mm or within a range of any two of these values.
A19. The devices or methods of any prior methods, wherein the ratio of total area of wells on plate to store liquid reagent to total area of plate is 1% or less, 2% or less, 5% or less, 10% or less, 15% or less, 20% or less, 30% or less, 40% or less, 50% or less, 60% or less, 70% or less, 80% or less, 90% or less, 95% or less, 99% or less or within a range of any two of these values.
A20. The devices or methods of any prior methods, wherein the liquid reagents stored in wells is one type of the reagents.
A21. The devices or methods of any prior methods, wherein the liquid reagents stored in wells are several types of the reagents in different wells.
A22. The devices or methods of any prior methods, wherein the plates with stored liquid reagent has hydrophilic surface.
A23. The devices or methods of any prior methods, wherein the plates without stored liquid reagent has hydrophobic surface.
A24. The devices or methods of any prior methods, wherein the sealing film for storing liquid reagent has hydrophobic surface.
A25. The devices or methods of any prior methods, wherein the sealing film has a thickness of 5 um, 10 um, 20 um, 30 um, 50 um, 100 um, 200 um, 300 um, 500 um, or within a range of any two of these values.
A26. The devices or methods of any prior methods, wherein the materials of the sealing film are selected from polystyrene, PMMA, PC, COC, COP, or another plastic.
A27. The devices or methods of any prior methods, wherein the liquid reagents on plates are coated by, for example, droplet printing, ink jet printing, air jet printing, transfer printing from a stamp, or a combination thereof.

### 1. Device for Composite Liquid Sample Separation

In one aspect, the present invention provides a device for separating a component from a composite liquid sample, comprising: a collection plate having a plurality of pillar spacers on one of its surfaces, and a filter having a sample receiving surface and a sample exit surface, wherein at least a part of the pillar spacers of the collection plate contact with and point against the sample exit surface, forming micro-cavities confined by the sample exit surface and the part of the pillar spacers, wherein the micro-cavities provide a capillary force that is at least a first part of a driving force for causing at least a part of the sample that is deposited on the sample receiving surface to flow through the filter toward the collection plate, and wherein the filter is configured to separate said component from said part of the sample.

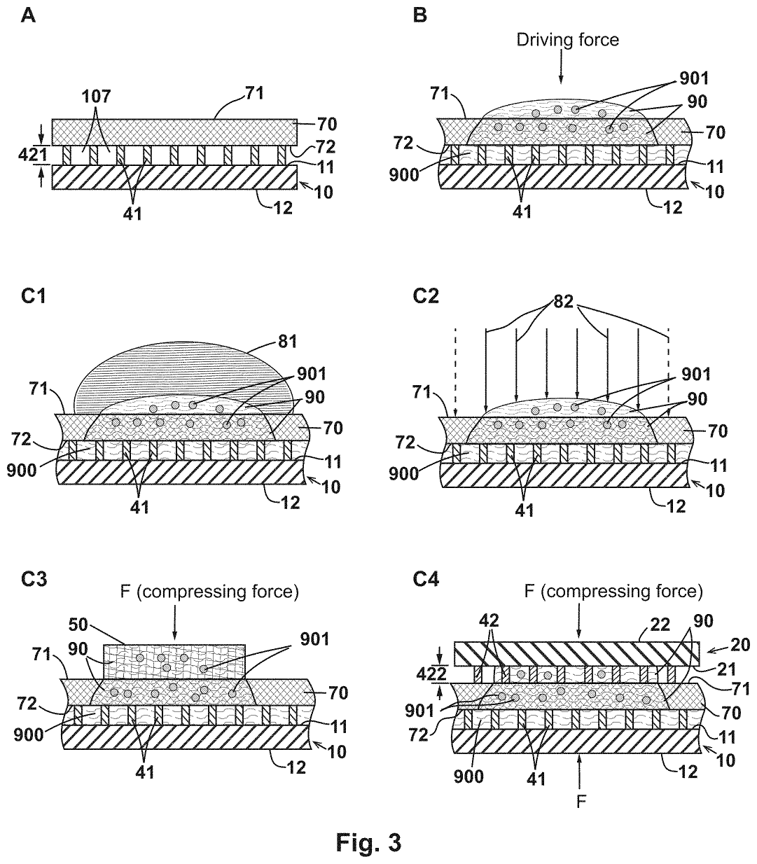

FIG. 3 panel (A) illustrates one exemplary embodiment of the device, where the device comprises a collection plate 10 and a filter 70. As shown in panel (A), in some embodiments, the collection plate 10 has an inner surface 11, an outer surface 12, and a plurality of pillar spacers 41 on its inner surface 11. The filter 70 has a sample receiving surface 71 and a sample exit surface 72. In some embodiments, the pillar spacers 41 are fixed on the inner surface 11. At least a part of the pillar spacers 41 point against and be in contact with the sample exit surface 72 of the filter 70, forming microcavities (not shown) or micropores in the filter media 70 that are confined by the sample exit surface 72 and part of the pillar spacers 41.

FIG. 3 panel (B) further illustrates an exemplary embodiment of the device, where a composite liquid sample 90 containing a component 901 to be removed, is deposited on the sample receiving surface 71 of filter 70. According to one aspect of the present invention, the filter 70 is configured to separate the component 901 from the part of the sample 90 as it flows through the filter 70 from the sample receiving surface 71 toward the collection plate 10. As shown in panel (B), in some embodiments, at least a part of the sample 90 is driven by a driving force to flow through the filter 70, in a direction from the sample receiving surface 71 toward the sample exit surface 72 and the collection plate 10. As the part of the sample 90 flows through the filter 70, the component 901 is retained and/or removed by the filter 70 from the filtered product 900 (i.e., the filtrate) the part of the sample that exits the filter 70. In some embodiments, the microcavities in the filter media 70 and/or the filter 70 provide a capillary force that is at least a part of the driving force. In some embodiments, the capillary force the microcavities in the filter media 70 and/or the filter 70 provide is the only and the entire part of the driving force. However, in other embodiments, the capillary force from the microcavities and/or the filter 70 is only a part of, sometimes even a negligible part of, the driving force.

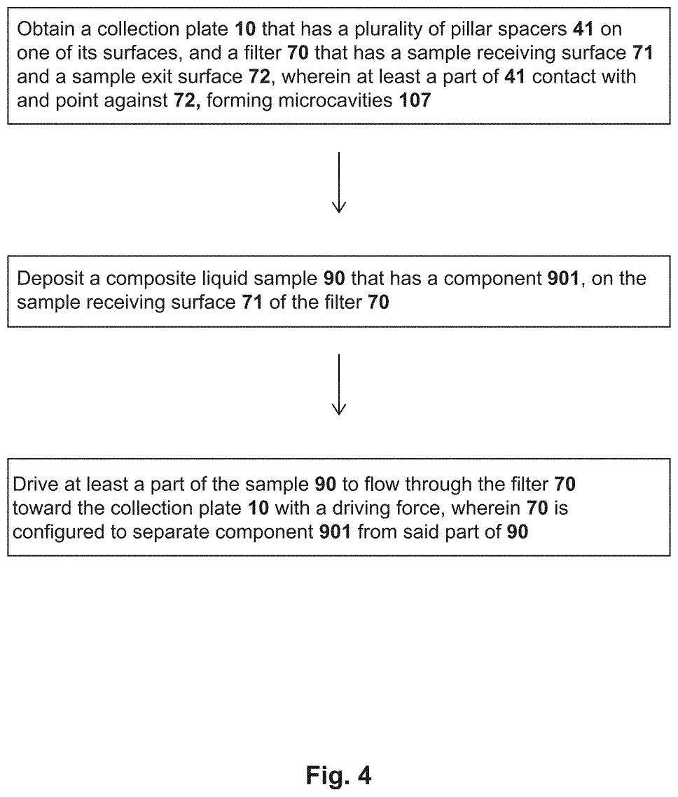

The features for the common device, shown in FIG. 3 panels (A) and (B) and described, are also applicable to the embodiments shown in all the other panels in FIG. 3, and FIGS. 4 to 6 and described. In addition, it should be noted that the device serves as an example for the features shown in all figures and described thereof.

FIG. 3 panels (C1) to (C4) schematically show different embodiments of the disclosed device, where the device further comprises a source providing at least a part of the driving force for causing at least part of the sample 90 to flow through the filter 70 toward the collection plate 10. These exemplary sources disclosed herein are by no means meant to be exclusive as to other possible embodiments and combination of any these sources with other embodiments. These sources are deployed separately, alternatively, sequentially, or combinatorially, or in any other manner as long as it serves its main function, that is to provide at least a part of the driving force for causing the sample flow for the component separation by the filter 70.

As shown in FIG. 3 panel (C1), in some embodiments, the device further comprises a source (not shown) providing a first liquid 81 that has a low, if not zero, intermiscibility with the sample 90 and is configured to provide at least a part of the driving force. For instance, in situations where the sample 90 is a water-based solution, the first liquid 81 may be chosen from various types of hydrocarbon oils including, for example, mineral oil, gasoline and related petroleum products, vegetable oils, and any mixture thereof. In some embodiments, the first liquid 81 has higher density than the sample 90 and it drives the sample flow by its own gravity. In some embodiments, the first liquid 81 experiences a larger capillary force provided by the microcavities in the filter media 70 and/or the filter 70 and consequently is capable of driving the sample 90 to flow. In other embodiments, the first liquid 81 is pressurized and the pressure is applied against the filter 70 and the collection plate 10, therefore forcing the sample 90 to flow toward the collection plate 10. In yet other embodiments, the first liquid 81 has high intermiscibility with the sample 90, as long as it is configured to drive a part of the sample 90 to flow through the filter 70, for instance it can be highly pressurized. However, it should be noted that this type of configuration may compromise the quality of the filtered product 900, for instance, the filtered product 900 may be contaminated by the first liquid 81, and thus the analyte in the filtered product 900 may be diluted and/or altered physically or chemically by the contaminating first liquid 81, which may not be desirable in most applications.

As shown in FIG. 3 panel (C2), in some embodiments, the device further comprises a source (not shown) providing a pressured gas 82 that is configured to provide at least a part of the driving force. As illustrated, in some embodiments, the pressured gas 82 is applied against at least part of the sample 90 in the direction from the sample receiving surface 71 toward the sample exit surface 72.

In some embodiments, the device further comprises a sponge for providing at least a part of the driving force. The term “sponge” as used herein, refers to a flexible porous material that has pores with their shapes changeable under a force and that can absorb a liquid into the material or release a liquid out of the material, when the shape of the pores is changed. The sponge usually has an uncompressed state and a compressed state. In the uncompressed state, the porous structure of the sponge reaches its maximum internal dimension, that is the internal pores are in their largest shape having their highest possible volume therein in the absence of major external influences, while in the compressed state, in some embodiments, the sponge experiences an external compressing force, and consequently, the internal pores of the sponge are compressed and deformed to a shape with dimensions smaller than the maximum internal dimension. The major external influences refer to any external impact that deforms the internal pores of the sponge. When a sponge deforms in a direction from its compressed state to the uncompressed state, the sponge can absorb any liquid it is in fluid connection with; when the sponge deforms in an opposite direction, from its uncompressed state to the compressed state, the sponge releases the liquid it contains.

For example, FIG. 3 panel (C3) illustrates some embodiments of the device, where the device further comprises a sponge 50. As aforementioned, the sponge 50 has an uncompressed state and a compressed state. In some embodiments, the sponge 50 is relatively movable to the collection plate and the filter into different configurations:
(i) one of the configurations is a depositing configuration (not shown), in which: the sponge 50 is in the uncompressed state and separated, partially or completely, from the collection plate 10 and the filter 70, the distance between the collection plate 10 and the sponge 50 is not regulated by the spacers 41, the filter 70, or the deposited sample 90;
(ii) another of the configurations is a filtering configuration, in which: as shown in panel (C3), the filter 70 is positioned between the sponge 50 and the collection plate 10, the distance between the collection plate 10 and the sponge 50 is regulated by the spacers 41, the filter 70, and the deposited sample 90, the sponge 50 is in the compressed state, which is configured to provide at least a part of the driving force.

According to these embodiments, in the depositing configuration, the sponge 50 absorbs the liquid sample when placed in contact with the sample 90 so that a part or an entirety of the sample 90 enters the sponge 50 as shown in the figure. When the sponge 50, the collection plate 10, and the filter 70 are brought into their filtering configuration (i.e., the sponge 50 is compressed by a compressing force to its compressed state, and the distance between the collection plate 10 and the sponge 50 is regulated by the spacers 41, the filter 70, and the deposited sample 90), part of the absorbed sample 90 in the sponge 50 is forced to exit the sponge 50 and flow through the filter 70 toward the collection plate 10. Therefore, the component 901 is retained and/or removed from the filtered product 900 (i.e., filtrate). In some embodiments, the compressing force is applied on the sponge 50 in a direction against the filter 70. In other embodiments, the compressing force is applied on the sponge 50 in any other direction, so long as the sample 90 is forced to flow through the filter 70 toward the collection plate 10.

FIG. 3 panel (C4) shows yet other embodiments of the device, where the device further comprises a press plate 20, the press plate 20 having a plurality of spacers 42 on one of its surfaces. In some embodiments, the press plate 20 is relatively movable to the collection plate 10 and the filter 70 into different configurations:
(i) one of the configurations is a depositing configuration, in which the press plate 20 is separated, partially or completely, from the collection plate 10 and the filter 70, the distance between the collection plate 10 and the press plate 7 is not regulated by their spacers 41 and 42, the filter 70, or the deposited sample 90;
(ii) another of the configurations is a filtering configuration, in which: as shown in FIG. 3 panel (C4), the filter 70 is positioned between the press plate 20 and the collection plate 10, the distance between the collection plate 10 and the press plate 20 is regulated by their spacers 41 and 42, the filter 70, and the deposited sample 90, at least a part of the pillar spacers 42 and an inner surface 21 of the press plate press at least a part of the deposited sample 90 against the filter 70, providing at least a part of the driving force.

FIG. 3 panel (C4) shows that, in some embodiments, the collection plate 10, the filter 70, and the press plate 20 are brought into the filtering configuration by a compressing force that is applied over the press plate outer surface 22 and the collection plate outer surface 12. In the filtering configuration, the press plate pillar spacers 42 point against and are in contact with the filter 70 and at least part of the deposited sample 90. The distance between the press plate inner surface 11 and the sample receiving surface 71 is reduced to about the height of the pillar spacers 42. In some embodiments, in the filtering configuration of the device, at least a part of the deposited sample 90 is forced to flow through the filter 70 toward the collection plate 10, due to one of the following reasons, any combination thereof or any other alternatives: (a) the height of the pillar spacers 42 are configured to be smaller than the unconfined height of the deposited sample 90; (b) the filter 70 is configured to have a relatively low hindrance for the deposited sample 90 to flow through it in the direction from the sample receiving surface 71 toward the sample exit surface 72; (c) the microcavities (not shown) in the filter media 70 are configured to provide a relatively high capillary force to attract the sample flow toward the collection plate 10; and (d) the pillar spacer 42 are configured to provide a relatively high hindrance for the lateral flow of deposited sample 90.

### 1.1. X-Plate

In some embodiments of the present invention, the collection plate is also termed “X-plate”. It is a plate that comprises, on its surface, (i) spacers that have a predetermined inter-spacer distance and a predetermined height and are fixed on the surface, and (ii) a sample contact area for contacting a sample to be deposited, wherein at least one of the spacers is inside the sample contact area.

In some embodiments, the press plate is also a “X-plate”. Therefore, in these embodiments, the press plate, the filter, and the collection plate, in the filtering configuration of the device, become a sandwich-like structure, with the filter being compressed in the center by the two X-plates.

The details of the X-plates are pre-determined to provide appropriate parts of the driving force for causing the deposited sample to flow through the filter from the press plate side to the collection plate side, including, but not limited to, the thickness, shape and area, flexibility, surface flatness and wetting properties of the plate, the height, lateral dimension, interspace of the pillar spacers, the material and mechanical strength of the plate and pillar spacers.

In some embodiments, the X-plate includes, but not limited to, the embodiments described in U.S. Provisional Patent Application No. 62/202,989, which was filed on Aug. 10, 2015, U.S. Provisional Patent Application No. 62/218,455, which was filed on Sep. 14, 2015, U.S. Provisional Patent Application No. 62/293,188, which was filed on Feb. 9, 2016, U.S. Provisional Patent Application No. 62/305,123, which was filed on Mar. 8, 2016, U.S. Provisional Patent Application No. 62/369,181, which was filed on Jul. 31, 2016, U.S. Provisional Patent Application No. 62/394,753, which was filed on Sep. 15, 2016, PCT Application (designating U.S.) No. PCT/US2016/045437, which was filed on Aug. 10, 2016, PCT Application (designating U.S.) No. PCT/US2016/051775, which was filed on Sep. 14, 2016, PCT Application (designating U.S.) No. PCT/US2016/051794, which was filed on Sep. 15, 2016, and PCT Application (designating U.S.) No. PCT/US2016/054025, which was filed on Sep. 27, 2016; all of these disclosures are incorporated by reference for their entirety and for all purposes.

### 1.2. Filter

The term “filter”, as used herein, refers to a device that has at least a sample receiving surface and a sample exit surface, and that eliminates a certain component from a composite liquid sample, when the liquid sample flows through the filter in a direction that traverses both the first and sample exit surfaces. According to one aspect of the present invention, the filter can be a mechanical, chemical, or biological filter, or any combination thereof.

In some embodiments of the present invention, the filter can be a mechanical filter. Mechanical filter mechanically eliminates, trapping or blocking, certain solid components from a composite liquid sample when the sample flows through the filter in a certain direction. It is typically made of porous material, whereas the pore size determines the size of the solid particles capable of flowing through the filter and the size of the solid particle being eliminated from the sample that flows through it. The components of mechanical means are inert, so that they do not affect or interfere with the sample. Examples of a mechanical filter include, but not limited to, foam (reticulated and/or open cell), fibrous material (e.g., filter paper), gel, sponge, and like materials. Examples of materials include cellulose acetate, cellulose esters, nylon, polytetrafluoroethylene polyester, polyurethane, gelatin, agarose, polyvinyl alcohol, polysulfone, polyester sulfone, polyacrylonitrile, polyvinylidiene fluoride, polypropylene, polyethylene, polyvinyl chloride, polycarbonate, any other materials that can form a porous structure and any combination thereof.

In some embodiments of the present invention, the pore size of the mechanical filter is uniform or vary in a range with a pre-determined distribution. In some embodiments, the average pore size of the mechanical filter is 10 nm, 20 nm, 40 nm, 80 nm, 100 nm, 200 nm, 400 nm, 800 nm, 1 μm, 2 μm, 4 μm, 8 μm, 10 μm, 20 μm, 40 μm, 80 μm, 100 μm, 500 μm, 1 mm to 1 cm, 5 mm, or a range between any of the values.

In some embodiments of the present invention, the filter is a chemical filter, which chemically eliminates certain components from a composite liquid sample when the sample flows through it in a certain direction. In some embodiments, it comprises a chemical reactant and a housing for the chemical reactant. The chemical reactant specifically reacts with certain component that is to be eliminated from the sample. It is capable of binding and immobilizing the component, or converting the component to other material(s) that is/are either retained in the housing or released outside of the housing and the filtering product. In some embodiments, the chemical reactant is an inorganic chemical, organic chemical, or any combination thereof. In some embodiments, the chemical reactant can be biological material, including, for example, an antibody, an oligonucleotide, and other biological macromolecules that have affinity to the component that is to be eliminated from the sample.

In some embodiments of the present invention, the filter can also be a biological filter. A biological filter comprises a biological living matter and a housing for the living matter. In some embodiments, the living matter specifically ingests, engulfs, or binds to and immobilizes a certain component in the sample. Exemplary living matter that can be used in the biological filter include, for example, bacteria, fungus, virus, mammalian cells that have engulfing functions or affinity-binding properties, like a macrophage, T-cell, B-cell, and like entities.

### 2. Method for Composite Liquid Sample Separation

In one further aspect, the present invention provides a method for composite liquid sample separation, comprising the steps of:
(1) providing a collection plate having a plurality of pillar spacers on one of its surfaces, and a filter having a sample receiving surface and a reverse sample exit surface, wherein at least a part of the pillar spacers of the collection plate are in contact with and point against the sample exit surface, forming micro-cavities confined by the sample exit surface and said part of the pillar spacers of the collection plate;
(2) depositing the sample on the sample receiving surface of the filter; and
(3) driving at least a part of the deposited sample to flow through the filter toward the collection plate with a driving force, wherein the filter is configured to separate the component from the part of the deposited sample, and wherein at least a first part of the driving force is a capillary force provided by the micro-cavities.

FIG. 4 is a flow chart for an exemplary embodiment of the disclosed method. In this embodiment, the exemplary device as shown in FIG. 3 panel (A) is used. First, a user of the device obtains a collection plate 10 having a plurality of pillar spacers 41 on one of its surfaces, and a filter 70 having a sample receiving surface 71 and a sample exit surface 72, wherein at least a part of the pillar spacers 41 contact with and point against the sample exit surface 72, forming microcavities, which are confined by the sample exit surface 72 and the collection plate 10 (600). Next, depositing the composite liquid sample 90, having a component 901 to be separated from the sample, on the sample receiving surface 71 of the filter 70 (610). After the depositing step, driving at least a part of the sample 90 to flow through the filter 70 toward the collection plate 10 with a driving force, wherein the filter 70 is configured to separate component 901 from part of 90 (620), resulting in the filter product 900 (i.e., filtrate), and wherein the microcavities are configured to provide a part of the driving force.

In some embodiments, the part of the driving force that the microcavities provide is an entirety of the driving force. In these embodiments, the driving step is indeed to let the microcavities draw the part of sample 90 toward the collection plate 10 via capillary force, without any need of external influences.

In other embodiments, the part of the driving force that the microcavities provide is only a part thereof, such that another source is needed to provide the other part of the driving force. For instance, in some embodiments, gravity participates in the process of driving the sample 90 to flow through the filter 70 such as when the sample receiving surface 71 is facing upward toward the sky compared to the sample exit surface 72 and the collection plate 10 facing toward the earth. Or in other cases, another source is part of the device as provided above, including, for example, a source providing a first liquid 81, a source providing a pressured gas 82, a sponge 50, and a press plate 20. The driving force provided by these sources, including gravity, can be exploited separately, alternatively, sequentially, or combinatorially, or in any other manner as long as to serve their main function to provide at least a part of the driving force for causing the sample flow for the component separation by filter 70. According to these embodiments, the driving step of the method further comprises providing and operating the source for providing at least a part of the driving force.

In some embodiments, the driving step of the method comprises depositing a first liquid to contact the deposited sample, the first liquid having low intermiscibility with the sample and configured to provide at least a part of the driving force.

In other embodiments, the driving step of the method comprises applying a pressurized gas against the deposited sample, the pressurized gas being configured to provide at least a part of the driving force.

In other embodiments, the driving step of the method comprises: (a) contacting a sponge with the deposited sample; (b) compressing the sponge against the filter to provide at least a part of the driving force.

In yet other embodiments, the driving step of the method comprises: (a) placing a press plate, having a plurality of pillar spacers on one of its surfaces, to contact with the deposited sample, wherein at least a part of the pillar spacers of the press plate point against the sample receiving surface of the filter and are in contact with the deposited sample; (b) after the placing step (a), compressing the press plate against the filter to reduce the distance between the press plate and the filter, and to provide at least a part of the driving force.

### 3. Method for Assaying and the Sample after Separation and Reading

In one further aspect, the present invention provides a method for assaying the sample after separation and reading, comprising the steps of:
(1) providing a collection plate having a plurality of pillar spacers on one of its surfaces, and a filter having a sample receiving surface and a reverse sample exit surface, wherein at least a part of the pillar spacers of the collection plate are in contact with and point against the sample exit surface, forming micro-cavities confined by the sample exit surface and the part of the pillar spacers of the collection plate;
(2) depositing the sample on the sample receiving surface of the filter; and
(3) driving at least a part of the deposited sample to flow through the filter toward the collection plate with a driving force, wherein the filter is configured to separate the component from the part of the deposited sample, and wherein at least a first part of the driving force is a capillary force provided by the micro-cavities;
(4) the component from part of the deposited sample reacts with a reagent coated in the micro-cavities; and
(5) detecting and analyzing the signal from micro-cavities by image or lump sum optical signal.

The method of any prior embodiment, wherein one or both plate sample contact surfaces comprise one or a plurality of storage sites that each stores a reagent or reagents, wherein the reagent(s) dissolve and diffuse in the sample during or after step (3).

The method of any prior embodiment, wherein one or both plate sample contact surfaces comprise one or a plurality of storage sites that each stores beads, wherein the beads dissolve and diffuse in the sample during or after step (3).

The method of any prior embodiment, wherein one or both plate sample contact surfaces comprises one or a plurality of amplification sites that are each capable of amplifying a signal from the analyte or a label of the analyte when the analyte or label is within 500 nm from an amplification site.

The devices or methods of any prior embodiment, wherein the device further comprises, on one or both plates, a releasable dry reagent and a time-controlled release material that delays the time that the releasable dry regent is released into the sample.

The devices or methods of any prior embodiment, wherein the releasable dry reagent is a labeled reagent.

The devices or methods of any prior embodiment, wherein the releasable dry reagent is an enzymatic colorimetric reagent.

The devices or methods of any prior embodiment, wherein the releasable dry reagent is a fluorescently-labeled reagent.

The devices or methods of any prior embodiment, wherein the releasable dry reagent is a fluorescently-labeled antibody.

The devices or methods of any prior embodiment, wherein the releasable dry reagent is a cell stain.

The devices or methods of any prior embodiment, wherein the releasable dry reagent is a cell lysing.

The device, kit, system, smartphone system, and method of any prior embodiments, wherein the beads are prepared by:

(a) activating with N-Hydroxysuccinimide (NHS);

(b) blocking with a BSA solution; and

(c) incubating with a capture agent solution.

The devices or methods of any prior embodiment, wherein reaction in micro-cavities is an immunoassay.

The devices or methods of any prior embodiment, wherein reaction in micro-cavities is a colorimetric assay.

The devices or methods of any prior embodiment, wherein reaction in micro-cavities is a nucleic acid hybridization assay.

The devices or methods of any prior embodiment, wherein reaction in micro-cavities is a nucleic acid amplification assay.

The devices or methods of any prior embodiment, wherein reaction in micro-cavities is a beads aggregation/de-aggregation assay.

The devices or methods of any prior embodiment, wherein the detector is an optical detector that detects an optical signal.

The devices or methods of any prior embodiment, wherein the detector is an electric detector that detect electrical signal.

In assaying, a manipulation of a sample or a reagent can lead to improvements in the assay. The manipulation includes, for example, manipulating the geometric shape and location of a sample and/or a reagent, a mixing or a binding of a sample and a reagent, and a contact area of a sample of reagent to a plate

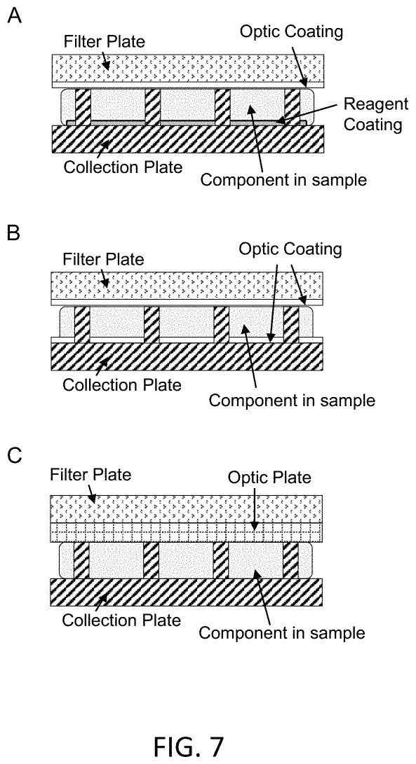

FIG. 7 shows schematics A to C where (A) exemplary device with a collection plate having a plurality of pillar spacers on one of its surfaces and coated with a reagent on its inner surface, and a filter plate having a sample receiving surface and a sample exit surface, wherein at least a part of the pillar spacers contact with and point against the sample exit surface, forming microcavities, which are confined by the sample exit surface and the collection plate; an optic coating is on the inner surface of the filter for helping the imaging and reading of the assay signal. (B) exemplary device with a collection plate having a plurality of pillar spacers on one of its surfaces, and a filter having a sample receiving surface and a sample exit surface, wherein at least a part of the pillar spacers contact with and point against the sample exit surface, forming microcavities, which are confined by the sample exit surface and the collection plate; optic coatings are on both of the inner surfaces of the filter and collection plate for helping the imaging and reading of the assay signal. (C) is an exemplary device with a collection plate having a plurality of pillar spacers on one of its surfaces, and a filter having a sample receiving surface and a sample exit surface, wherein at least a part of the pillar spacers contact with and point against the sample exit surface, forming microcavities, which are confined by the sample exit surface and the collection plate; an optic plate with passing holes is underneath the filter for helping the imaging and reading of the assay signal.

The method of any prior embodiment, wherein:

i. one or both plate sample contact surfaces comprise one or a plurality of binding sites that each bind and immobilize a respective analyte; or

ii. one or both plate sample contact surfaces comprise, one or a plurality of storage sites that each stores a reagent or reagents; wherein the reagent(s) dissolve and diffuse in the sample during or after step (c), and wherein the sample contains one or plurality of analytes; or

iii. one or a plurality of amplification sites that are each capable of amplifying a signal from the analyte or a label of the analyte when the analyte or label is 500 nm from the amplification site; or

iv. any combination of i to iii.

In one embodiment, reagent and additives are coated on the same plate (collection or filter plate), or a separate plate (i.e., collection or filter plate).

The device, kit, system, or method of any prior embodiments, wherein the reagents is coated by droplet printing into an array.

The device, kit, system, or method of any prior embodiments, wherein the reagents is coated by spray.

The device, kit, system, or method of any prior embodiments, wherein the reagents is coated by contact printing.

The device, kit, system, or method of any prior embodiments, wherein the reagents is coated by transfer printing.

In one embodiment, an optical coating is on the inner side of filter for helping the imaging and reading of the assay signal.

In one embodiment, an optical coating is on the inner side of collection plate for helping the imaging and reading of the assay signal.

In one embodiment, optical coatings are on both the inner side of collection plate and filter plate for helping the imaging and reading of the assay signal.

In one embodiment, optical coatings have the function of isolation the optical signal from one side of the coating.

In one embodiment, optical coatings have the function of isolation and blocking the red color from blood cells when filtering the plasma from blood.

In one embodiment, optical coatings have the function of optical reflection, amplification, filtration, polarization, and diffusion the optical signal from one side.

In one embodiment, optical coatings are a thin metallic film as gold, silver, aluminum, and like metallics.

In one embodiment, the optical coatings have a preferred thickness of 5 nm, 10 nm, 50 nm, 100 nm, 300 nm, 500 nm, 1 um, or in a range between any of the two values. In one embodiment, the optical coatings have a preferred thickness of 1 um, 2 um, 5 um, 10 um, 20 um, 50 um or in a range between any of the two values.

In one embodiment, an optical plate or film is on the inner side of filter for helping the imaging and reading of the assay signal.

In one embodiment, an optical plate or film is on the inner side of collection plate for helping the imaging and reading of the assay signal.

In one embodiment, optical plates or films are on both the inner side of collection plate and filter plate for helping the imaging and reading of the assay signal.

In one embodiment, the optical plates have holes on it to let the liquid flowing through.

In one embodiment, optical plates have the function of isolation the optical signal from one side of the coating.

In one embodiment, optical plates have the function of isolation and blocking the red color from blood cells when filtering the plasma from blood.

In one embodiment, optical plates have the function of optical reflection, amplification, filtration, polarization and diffusion the optical signal from one side.

In one embodiment, optical plates contain a thin metallic film such as gold, silver, aluminum and like metallics.

In one embodiment, the optical plates have a preferred thickness of 500 nm, 1 um, 2 um, 10 um, 20 um, 30 um, 50 um or in a range between any of the two values.

The reagent and application in an embodiment can include, for example:

A. Glucose Colorimetric (Fluorometric) assay
Sample: Whole Blood, Plasma, Serum, Saliva
Reagent Recipe 1: Glucose Oxidase, 100 unit/ml; Horseradish Peroxidase, 100 unit/ml; 4-amino antipyrine, 20 mM; and TOOS, 20 mM
Reagent Recipe 2: Glucose Oxidase, 100 unit/ml; Horseradish Peroxidase, 100 unit/ml; and 3,5,3′,5′-Tetramethylbenzidine (TMB), 20 mM
Reagent Recipe 3: Glucose Oxidase, 100 unit/ml; Horseradish Peroxidase, 100 unit/ml; and Amplex Red, 20 mM
Reagent Recipe 4: Hexokinase, 1 unit/ml; ATP, 220 μg/ml; and NAD, 400 μg/ml

B. Calcium Colorimetric assay
Sample: Whole Blood, Plasma, Serum, Saliva
Reagent Recipe 1: Arsenazo III, 17 μg/ml

C. Albumin Colorimetric assay
Sample: Whole Blood, Plasma, Serum, Saliva
Reagent Recipe 1: Bromocresol purple, 22 μg/ml

D. Total Protein Colorimetric assay
Sample: Whole Blood, Plasma, Serum, Saliva
Reagent Recipe 1: Cupric sulfate, 1.34 mg/ml; Sodium potassium tartrate, 3.43 mg/ml; and Potassium iodide, 0.28 mg/ml

E. Sodium Colorimetric Assay
Sample: Whole Blood, Plasma, Serum, Saliva
Reagent Recipe 1: ONPG, 220 μg/ml; and β-Galactosidase, 0.05 unit/ml

F. Potassium Colorimetric assay
Sample: Whole Blood, Plasma, Serum, Saliva
Reagent Recipe 1: ADP, 220 μg/ml; Phosphoenolpyruvate, 0.05 unit/ml; Pyruvate kinase, 0.1 unit/ml; NADH, 480 μg/ml; Potassium phosphate, 13.6 mg/ml; Magnesium sulfate, 95 μg/ml; FAD, 7.85 μg/ml; 4-Aminoantipyrine, 130 μg/ml; Horseradish Peroxidase, 10 unit/ml; and TBHBA, 1.88 mg/ml

G. Chloride Colorimetric assay
Sample: Whole Blood, Plasma, Serum, Saliva
Reagent Recipe 1: CNPG3, 530 μg/ml; α-Amylase, 0.36 unit/ml; and Calcium acetate, 250 μg/ml

H. Blood Urea Nitrogen Colorimetric assay
Sample: Whole Blood, Plasma, Serum, Saliva
Reagent Recipe 1: Urea Amidolyase, 0.5 U/ml; PEP, 570 ug/ml; ATP, 220 ug/ml; Pyruvate Kinase, 1 U/ml; Pyruvate Oxidase, 10 U/ml; Potassium phosphate, 13.6 mg/ml; MgCl2, 95 ug/ml; FAD, 7.85 ug/ml; TBHBA, 1.88 mg/ml; 4-AAP, 130 ug/ml; and Peroxidase, 10 U/ml

I. Creatinine Colorimetric assay
Sample: Whole Blood, Plasma, Serum, Saliva
Reagent Recipe 1: Creatinine Amidohydrolase, 10 U/ml; Creatinine Amidinohydrolase, 30 U/ml; Sarcoosine Oxidase, 10 U/ml; TBHBA, 1.88 mg/ml; 4-AAP, 130 ug/ml; and Peroxidase, 10 U/ml

J. Alkaline Phosphatase Colorimetric assay
Sample: Whole Blood, Plasma, Serum, Saliva
Reagent Recipe 1: p-Nitrophenyl Phosphate, 560 ug/ml; Zinc Sulfate, 0.5 U/ml; and Magnesium Sulfate, 330 ug/ml

K. Alanine Amino Transferase Colorimetric assay
Sample: Whole Blood, Plasma, Serum, Saliva
Reagent Recipe 1: L-Alanine, 8.74 mg/ml; α-Ketoglutaric Acid, 1.01 mg/ml; Pyruvate Oxidase, 10 U/ml; Potassium phosphate, 13.6 mg/ml; MgCl₂, 95 ug/ml; FAD, 7.85 ug/ml; TBHBA, 1.88 mg/ml; 4-AAP, 130 ug/ml; and Peroxidase, 10 U/ml

L. Aspartate Amino Transferase Colorimetric assay
Sample: Whole Blood, Plasma, Serum, Saliva
Reagent Recipe 1: L-Aspartic Acid, 4.26 mg/ml; α-Ketoglutaric Acid, 1.01 mg/ml; Oxaloacetate decarboxylase, 10 U/ml; TBHBA, 1.88 mg/ml; 4-AAP, 130 ug/ml; and Peroxidase, 10 U/ml

M. Bilirubin Colorimetric assay
Sample: Whole Blood, Plasma, Serum, Saliva
Reagent Recipe 1: Bilirubin Oxidase, 1 U/ml

N. Cholesterol Colorimetric (Fluorimetric) assay
Sample: Whole Blood, Plasma, Serum, Saliva
Reagent Recipe 1: Cholesterol Oxidase, 100 unit/ml; Horseradish Peroxidase, 100 unit/ml; 4-amino antipyrine, 20 mM; and TOOS, 20 mM
Reagent Recipe 2: Cholesterol Oxidase, 100 unit/ml; Horseradish Peroxidase, 100 unit/ml; and 3,5,3′,5′-Tetramethylbenzidine (TMB), 20 mM
Reagent Recipe 3: Cholesterol Oxidase, 100 unit/ml; Horseradish Peroxidase, 100 unit/ml; and Amplex Red, 20 mM

O. Triglycerides Colorimetric (Fluorimetric) assay
Sample: Whole Blood, Plasma, Serum, Saliva
Reagent Recipe 1: Lipase, 100 unit/ml; Glycerokinase, 100 unit/ml; Glycerophosphate Oxidase, 100 unit/ml; 4-amino antipyrine, 20 mM; and TOOS, 20 mM
Reagent Recipe 2: Lipase, 100 unit/ml; Glycerokinase, 100 unit/ml; Glycerophosphate Oxidase, 100 unit/ml; and 3,5,3′,5′-Tetramethylbenzidine (TMB), 20 mM
Reagent Recipe 3: Lipase, 100 unit/ml; Glycerokinase, 100 unit/ml; Glycerophosphate Oxidase, 100 unit/ml; and Amplex Red, 20 mM

P. Alcohol Colorimetric (Fluorimetric) assay
Sample: Whole Blood, Plasma, Serum, Saliva, Breath
Reagent Recipe 1: Alcohol Oxidase, 100 unit/ml; Horseradish Peroxidase, 100 unit/ml; 4-amino antipyrine, 20 mM; and TOOS, 20 mM
Reagent Recipe 2: Alcohol Oxidase, 100 unit/ml; Horseradish Peroxidase, 100 unit/ml; 3,5,3′,5′-Tetramethylbenzidine (TMB), 20 mM
Reagent Recipe 3: Alcohol Oxidase, 100 unit/ml Horseradish Peroxidase, 100 unit/ml; Amplex Red, 20 mM

Q. Hydrogen Peroxide (Fluorimetric) assay
Sample: Whole Blood, Plasma, Serum, Saliva, Breath
Reagent Recipe 1: Horseradish Peroxidase, 100 unit/ml; 4-amino antipyrine, 20 mM; and TOOS, 20 mM
Reagent Recipe 2: Horseradish Peroxidase, 100 unit/ml; 3,5,3′,5′-Tetramethylbenzidine (TMB), 20 mM
Reagent Recipe 3: Horseradish Peroxidase, 100 unit/ml; and Amplex Red, 20 mM

R. Gram Staining
Sample: Blood smear, Vaginal samples, Genital samples
Gram Crystal Violet: Crystal Violet, 20 g; Ammonium Oxalate, 8 g; and Methanol, 200 mL
Gram Iodine: Iodine Crystal, 3.33 g; and Potassium Iodide, 6.67 g
Gram Decolorizer: Ethanol, 500.0 mL; and Acetone, 500.0 mL
Gram Safranin: Safranin O, 0.25 g; and Ethanol, 10 mL
Gram Basic Fuchsin Basic: Fuchsin, 0.7 g; Phenol, 3.5 mL; and Ethanol, 14 mM

S. Leishman Staining
Sample: Smear sample
Recipe Leishman's dye, 0.2 g; and Acetone-free methyl alcohol, 100 mL

T. Giemsa Staining
Sample: Smear sample
Recipe: Giemsa powder, 0.15 g; Glycerin, 12.5 mL; and Methyl alcohol, 12.5 mL

U. Wright Staining
Sample: Smear sample
Recipe: Wright stain, 1.5 g; and Methanol, 500 mL

V. Field Staining
Sample: Smear sample
Field Solution A: Methylene Blue, 1.6 g; Disodium dihydrogen phosphate, 10 g; Potassium dihydrogen phosphate, 12.5 g; Azur, 1 g; and Distilled water, 1000 mL
Field Solution B: Eosin Y, 2 g; Disodium dihydrogen phosphate, 10 g; Potassium dihydrogen phosphate, 12.5 g; Distilled water, 1000 mL

W. Jenner Staining
Sample: Smear sample
Recipe: Jenner stain, 0.5 g; and Methanol, 100 mL

X. JSB Staining
Sample: Smear sample
Recipe: Atine orange dye, 0.5 g; 1% Sulfuric acid, 3 mL; Potassium dichromate, 0.5 g; Disodium hydrogen phosphate dihydrate, 3.5 g; and Distilled water, 500 mL
JSB stain II: Eosin Y, 1 g; and Distilled water, 500 mL

Y. White Blood cells staining for counting and differentiation
Sample: Blood, urine, other body fluids
Recipe I: Acridine Orange (Detection agents), 1 ug/mL to 1 mg/mL
Recipe II: Propidium Iodide (PI) (Detection agents), 150 uM; Fluorescein Isothiocyanate (FITC), 100 uM; and Basic Orange 21 (B021) dye, 250 uM

Z. Platelets staining for counting
Sample: Blood, urine, other body fluidics
Recipe I: Acridine Orange (Detection agents), 1 ug/mL to 1 mg/mL
Recipe II: Propidium Iodide (PI) (Detection agents), 150 uM; Fluorescein Isothiocyanate (FITC), 100 uM; and Basic Orange 21 (B021) dye, 250 uM

### 4. Image-Based Reading Method and Imperfection Removal

In one preferred embodiment and the method of any prior embodiment, the assay is read by imaging.

In one preferred embodiment and the method of any prior embodiment, the assay is read by imaging through red, green, and blue channels.

In one preferred embodiment and the method of any prior embodiment, the assay is read by imaging through a grey scale channel.

In one embodiment, the image is analyzed with a machine learning method.

In some embodiments, the imaging of the sample can be used, for example, to detect, distinguish, classify, revise and/or correct the following instances in biological and chemical assay application in the device: (1) at the edge of sample, (2) air bubble in the sample, (3) too small sample volume or too much sample volume, (4) sample under the spacer, (5) aggregated sample, (6) lysed sample, (7) over exposure image of the sample, (8) under exposure image of the sample, (8) poor focus of the sample, (9) optical system error, (10) card not closed, (11) wrong card, (12) dust in the card, (13) oil in the card, (14) dirty or out of the focus plane for a card, (15) card not in proper position inside the reader, (16) empty card, (17) manufacturing error in the card, (18) wrong card for another application, (19) dried sample, (20) expired card, (21) large variation of distribution of signal, (22) wrong sample, and combinations thereof.

In some embodiments, the imaging of the sample can be used to remove or revise the imperfection area in the sample.

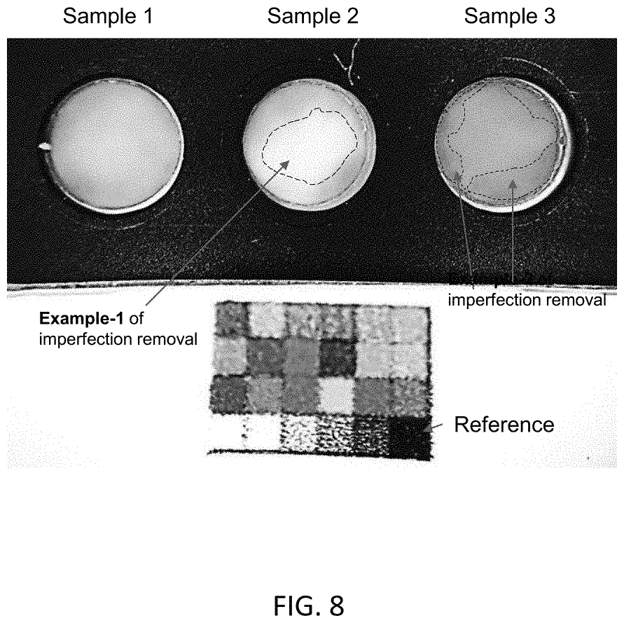

One example as shown in FIG. 8, where three colorimetric samples (on a vertical flow card measuring triglyceride in the whole blood) is read by color images. Sample 2 has central region with much lighter color intensity than surrounding areas due to an air bubble imperfection in the sample. Sample 3 has dark area imperfections near the well edges. Using an imaging method, these imperfection areas can be removed before the color intensity is analyzed to improve the accuracy of the assay. A color standard reference in the field of view can be used to calibrate the over image color.

In some embodiments, a reference in the image can be used to help the image analysis.

In some embodiments, the reference in the image can include, for example, a standard color calibrator, a contrast calibrator, a chemical reaction calibrator, a location calibrator, size calibrator, time calibrator, and like calibrators.

### 5. Sample

The composite liquid sample, according to one aspect of the present invention, comprises one or more components to be separated by the devices and methods provided from the sample.

The disclosed devices and methods can be used for samples such as a diagnostic sample, a clinical sample, an environmental sample, and a foodstuff sample. The types of sample can include, for example, the samples disclosed in PCT Application (designating U.S.) No. PCT/US2016/045437, filed on Aug. 10, 2016, and incorporated by reference by its entirety.

In particular embodiments, the sample can be obtained from a biological sample such as cells, tissues, bodily fluids, and stool. Typically, samples that are not in liquid form can be converted to liquid form before analyzing the sample with the disclosed methods. Bodily fluids of interest can include, for example, amniotic fluid, aqueous humour, vitreous humour, blood (e.g., whole blood, fractionated blood, plasma, serum), breast milk, cerebrospinal fluid (CSF), cerumen (earwax), chyle, chime, endolymph, perilymph, feces, gastric acid, gastric juice, lymph, mucus (including nasal drainage and phlegm), pericardial fluid, peritoneal fluid, pleural fluid, pus, rheum, saliva, sebum (skin oil), semen, sputum, sweat, synovial fluid, tears, vomit, urine, and exhaled condensate. In particular embodiments, a sample can be obtained from a subject, e.g., a human. In some embodiments, the sample can be processed prior to use in the subject assay. For example, prior to analysis, a protein/nucleic acid can be extracted from a tissue sample prior to use, using known extraction methods. In particular embodiments, the sample can be a clinical sample, e.g., a sample collected from a patient.

In particular embodiments, the sample can be obtained from an environmental sample source, including, for example: liquid samples from a river, lake, pond, ocean, glaciers, icebergs, rain, snow, sewage, reservoirs, tap water, drinking water, etc.; solid samples from soil, compost, sand, rocks, concrete, wood, brick, sewage, etc.; and gaseous samples from the air, underwater heat vents, industrial exhaust, vehicular exhaust, etc. Typically, samples that are not in liquid form are converted to liquid form before analyzing the sample with the disclosed method.

In particular embodiments, the sample can be obtained from a food sample that is suitable for animal consumption, e.g., human consumption. A foodstuff sample includes, for example, raw ingredients, cooked food, plant and animal sources of food, preprocessed food as well as partially or fully processed food, etc. Typically, samples that are not in liquid form are converted to liquid form before analyzing the sample with the present method.

According to one aspect of the present invention, the component(s) to be separated from the sample can be in a solid, a liquid, a gaseous state, or any combination thereof. The components to be separated from the sample include, for example, cells, tissues, virus, bacterium, proteins, DNAs, RNAs, gas bubbles, lipids.

In a preferred embodiment of the present invention, the sample can be a whole blood sample, and the components to be separated from the whole blood sample are blood cells (red blood cells, white blood cells, platelets, etc.). In a preferred embodiment, the devices and methods are particularly configured for plasma separation.

According to one aspect of the present invention, the sample volume can be 1 μL or less, 2 μL or less, 5 μL or less, 10 μL or less, 20 μL or less, 50 μL or less, 100 μL or less, 200 μL or less, 500 μL or less, 1 mL or less, 2 mL or less, 5 mL or less, 10 mL or less, 20 mL or less, 50 mL or less, 100 mL or less, 200 mL or less, 500 mL or less, 1 L or less, or a range between any of the values.

### 6. Filtered Product

In some embodiments of the present invention, the collection plate can be an X-plate, which, in addition to the composite sample separation, can be used in a QMAX process for further sensing/assaying/processing of the filtered product.

In the QMAX (Q: quantification; M: magnification; A: adding reagents; X: acceleration; also known as compressed regulated open flow (CROF)) process or assay or assay platform, a QMAX device uses two plates to manipulate the shape of a sample into a thin layer (e.g., by compressing).

In QMAX assays, one of the plate configurations is an open configuration, wherein the two plates are completely or partially separated (the spacing between the plates is not controlled by spacers) and a sample can be deposited. Another configuration is a closed configuration, wherein at least part of the sample deposited in the open configuration is compressed by the two plates into a layer of highly uniform thickness, the uniform thickness of the layer is confined by the inner surfaces of the plates and is regulated by the plates and the spacers.

In some embodiments of the present invention, after filtering the sample, the filter and the source providing the second part of the driving force are separated from the collection plate. The filtered product is retained on the collection plate, at least partially due to capillary force and surface tension. In some embodiments, the collection plate bearing the filtered product are joined with a capture plate to form a QMAX device. The collection plate and the capture plate are relatively movable to each other into different configurations, wherein one of the configurations is an open configuration, in which the collection plate and the capture plate are separated apart, the spacing between the plates is not regulated by the spacers, wherein another of the configurations is a closed configuration, in which the plates are facing each other, the spacers and the filtered product are between the plates, the thickness of the filtered product is regulated by the plates and the spacers and is thinner than that when the plates are in the open configuration, and at least one of the spacers is inside the sample.

In some embodiments of the present invention, the capture plate is a planar glass plate, and/or comprises a binding site or a storage site that contains a binding agent or a detection agent, respectively, for an assay of the filtered product. In some embodiments, the collection plate also comprises a binding site or storage site for an assay of the filtered product.

In some embodiments, the QMAX device that the collection plate and the capture plate form after the filtering process includes, for example, the embodiments described in U.S. Provisional Patent Application No. 62/202,989, filed Aug. 10, 2015, U.S. Provisional Patent Application No. 62/218,455, filed Sep. 14, 2015, U.S. Provisional Patent Application No. 62/293,188, filed Feb. 9, 2016, U.S. Provisional Patent Application No. 62/305,123, filed Mar. 8, 2016, U.S. Provisional Patent Application No. 62/369,181, filed Jul. 31, 2016, U.S. Provisional Patent Application No. 62/394,753, filed Sep. 15, 2016, PCT Application (designating U.S.) No. PCT/US2016/045437, filed Aug. 10, 2016, PCT Application (designating U.S.) No. PCT/US2016/051775, filed Sep. 14, 2016, PCT Application (designating U.S.) No. PCT/US2016/051794, filed Sep. 15, 2016, and PCT Application (designating U.S.) No. PCT/US2016/054025, filed Sep. 27, 2016; all of these disclosures are incorporated by reference for their entirety and for all purposes.

### 7. Advantageous Effects and Applications

The devices and methods provided by the present invention can have use in a variety of different applications in various fields, where separation of undesired components from a given composite liquid sample and/or extraction of desired components from a given sample are needed. For example, the subject device and method may find use in assays involving blood plasma where separation of blood cells is required, in applications requiring pure water without contaminating particles, in applications involving investigations of the contaminating bacterium in drinking water, and the like. The various fields include, for example, human, veterinary, agriculture, foods, environments, drug testing, and like fields.

The devices and methods provided in the present invention have many advantages over existing art for composite liquid sample separation for many reasons, including, for example: the devices and methods provided in some preferred embodiments can be relatively much simpler and easier to operate, void of the need for well-trained professionals, require a much shorter time, a much lower cost, and in some particular embodiments, are especially good at handling small volumes of liquid sample,

In addition, the devices provided in some preferred embodiments of the present invention can be used to form a QMAX device, which may find use in a wider range of applications. These applications include, for example, bio/chemical assays, quantitative sampling of liquid sample, bio/chemical processing, and biomarker detections.

The devices and methods disclosed have various types of biological/chemical sampling, sensing, assays, and applications, which include, for example, those described in PCT Application (designating U.S.) No. PCT/US2016/045437, filed Aug. 10, 2016, and PCT/US16/51794, filed Sep. 14, 2016; and are incorporated by reference in their entirety.

The devices and methods herein disclosed can be used for the detection, purification and/or quantification of analytes such as biomarkers. Examples of the biomarks can include what is disclosed in PCT Application (designating U.S.) No. PCT/US2016/045437, filed Aug. 10, 2016, and incorporated by reference in its entirety.

The disclosed devices and methods can used with the facilitation and enhancement of mobile communication devices and systems, which include devices and systems listed, described and summarized in PCT Application (designating U.S.) No. PCT/US2016/045437, filed Aug. 10, 2016, and is incorporated by reference in its entirety.

### 8. Example 1

Here exemplary devices and methods for separating plasma from a whole blood sample according to one aspect of the present invention have been achieved experimentally. Experiments have been carried out to test and compare different experimental conditions for plasma separation.

For this experiment, two different types of X-plates were used as the collection plate according to one aspect of the present invention. Both were made of PMMA and 175 μm thick and 1 inch by 1 inch wide. Type 1 X-plate has, on its surface, cubical pillar spacers of 30×40 μm in width and 30 μm in height and interspaced by 80 μm inter-spacing distance (ISD). Type 2 X-plate has, on its surface, cubical pillar spacers with all the same parameters as Type 1 except with 2 μm in height. In some experimental conditions, a different X-plate, chosen from one of the two types, was used as the press plate as well. Four types of filter membranes (all purchased from Sterlitech Corp., Kent, Wash. and made of polycarbonate) with different pore sizes (0.4 μm, μm, 1 μm, 2 μm, and 3 um) were used as the filter for separating the blood cells from the plasma in the blood sample.

Whole blood sample was obtained either commercially or freshly by pricking a human subject's finger. As for all experimental conditions, during plasma separation, a filter membrane was set on top of a collection plate, which was placed on a bench with its pillar spacers pointing upward, and then a drop of whole blood sample (1 μL when using a press plate with 2 μm high spacers and 3 μL when using a planar glass plate, sponge, or press plate with 30 μm high spacers) was deposited on top of the filter membrane for plasma separation. Either a planar glass plate, a sponge, or a press plate was used as the press media for providing the driving force for causing the blood sample to flow through the filter membrane toward the collection plate. The press media was placed on top of the deposited blood sample, and then hand-pressed against the collection plate for a certain amount of time (e.g., 30 or 180 seconds), thereby forcing the blood sample to flow through the filter membrane for plasma separation.

After the hand-pressing for plasma separation, the top press media and the filter membrane were peeled off, while the filtered product stayed on the collection plate. A different planar glass plate (“capture plate”, 1 mm thick and 1×1 inch wide) was then placed to contact the collection plate. Here, a QMAX process was then used for sample observation and quantitation. The collection plate and the capture plate were hand-pressed against each other for 30 second and then “self-held” to form a QMAX device. The resulting QMAX device bearing the filtered product was then imaged under light microscope, and the volume of the filtered product was estimated accordingly.

Eleven different experimental conditions were tested in this experiment and the details of each condition are summarized in Table 1.

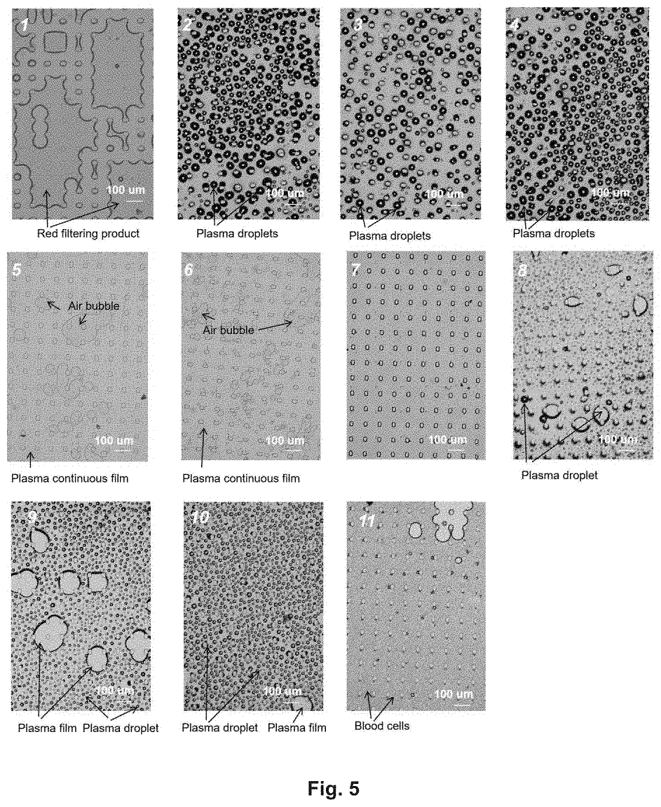

FIG. 5 shows the representative images of the filtered products that resulted from different experimental configurations of the device when used for plasma separation. The number (1 to 11) on the top left corner of each image denotes its experimental Group number as listed in Table 1, and the periodically arranged rounded rectangles shown in each image are the pillar spacers of the collection plates. As shown in the images, glass plates (Group 1) apparently lysed the red blood cells in the sample, leaving the filtered product in a visible red color. Group 11 showed blood cells in the filtered product, indicating that the pore size (5 um) was not small enough to filter out the blood cells. Group 7 showed little plasma or blood, likely due to the oversize of sponge, which absorbed and retained most, if not all, the blood sample. Plasma was obtained in all the other groups. As seen from the images, Groups 5 and 6 gave the best results as the filtered product (plasma) formed continuous films in the QMAX device. Groups 2, 3, 4, 8, 9, and 10 showed mainly plasma droplets and occasionally a few patchy plasma films, likely due to the 30 μm pillar height of the collection plate, as compared to the 2 μm pillar height in groups 5 and 6.

TABLE 1

| | Experimental Condition | | | | |
|---|---|---|---|---|---|
| | | | | | Blood |
| | | | Collection | Hand | sample |
| | | Filter | plate pillar | press | volume |
| | Press media | pore size | height | duration | (micro- |
| Group | (pillar height) | (microns) | (microns) | (seconds) | liters) |
| 1 | Glass | 0.4 | 30 | 30 | 3 μL |
| 2 | X-Plate (30 μm) | 0.4 | 30 | 30 | 3 μL |
| 3 | X-Plate (30 μm) | 0.4 | 30 | 180 | 3 μL |
| 4 | X-Plate (2 um) | 0.4 | 30 | 30 | 1 μL |
| 5 | X-Plate (2 um) | 0.4 | 2 | 30 | 1 μL |
| 6 | X-Plate (30 μm) | 0.4 | 2 | 30 | 3 μL |
| 7 | Sponge | 0.4 | 30 | 30 | 3 μL |
| 8 | X-Plate (30 μm) | 1.0 | 30 | 30 | 3 μL |
| 9 | X-Plate (30 μm) | 2.0 | 30 | 30 | 3 μL |
| 10 | X-Plate (30 μm) | 3.0 | 30 | 30 | 3 |
| 11 | X-Plate (30 μm) | 5.0 | 30 | 30 | 3 |

An estimation of the filtered product volume was performed by multiplying the height of the pillar spacers by the summed area of plasma calculated from the image, and the filtering efficiency was calculated by dividing the volume of the filtered product by the volume of the whole blood sample. The overall data is summarized in Table 2.

TABLE 2
Filtered product quantitation

Results

| | | Efficiency |
|---|---|---|
| | Filtering | (product/ |
| Group | product | whole blood) |
| 1 | ~1 μL | ~30% |
| | (with HbA) | |
| 2 | ~0.3 μL | ~10% |
| 3 | ~0.3 μL | ~10% |
| 4 | ~0.2 μL | ~20% |
| 5 | ~0.2 μL | ~20% |
| 6 | ~0.3 μL | ~10% |
| 7 | <0.1 μL | <3% |
| 8 | ~0.4 μL | ~13% |
| 9 | ~0.5 μL | ~17% |
| 10 | ~0.5 μL | ~17% |
| 11 | N/A | N/A |

This example illustrates the validity of the devices and methods provided by the present invention. It also demonstrates the advantages of using the present invention to realize plasma separation: the exemplary devices have relatively much simpler structure and are much easier to handle, as compared to many other existing arts in the field; the method takes much shorter time, likely within 1 min from obtaining the device and sample to the complete of the plasma separation; the method is capable of handling a very small amount of a blood sample, reduced burden on subjects, especially patients, by avoiding the invasive drawing of a large amount of blood.

### 9. Example-2

Here, the plasma separated by the exemplary device and method as illustrated in Example-1 has been demonstrated to be used for a triglyceride (TG) assay, a part of a regular lab test. TGs are a type of fat found in the blood, a high level of TGs may raise the risk of coronary artery disease. Therefore, a TG test is a part of a lipid panel that is used to evaluate an individual's risk of developing heart disease. Typically, a TG assay is a colorimetric assay and performed with plasma instead of whole blood sample to avoid color interference from hemoglobins in red blood cells. An exemplary device and method were used here to separate plasma from a whole blood sample, and the resulting plasma was used as a substrate for the TG assay.

In this experiment, for plasma separation, an X-plate (PMMA, 175 μm thick and 1×1 inch wide, cubical pillar spacers: 30×40 μm wide, 30 μm high, and 80 μm ISD) was used as the collection plate. A filter membrane with 0.4 μm pores (Sterlitech Corp., Kent, Wash.) was used as the filter. A different X-plate (PMMA, 175 μm thick and 1×1 inch wide, cubical pillar spacers: 30×40 μm wide, 30 μm high, and 80 μm ISD) was used as the press plate. About 2 μL whole blood sample was obtained freshly by pricking a subject's finger and deposited on the filter membrane, which was placed on top of the pillar spacers of the collection plate, and then the press plate was placed on top of the deposited sample and hand-pressed against the collection plate for 30 s. Part of the whole blood sample was forced to flow through the filter membrane toward the collection plate, realizing plasma separation.

For the TG assay, after plasma separation, the filter membrane and the press plate were then peeled off from the collection plate, leaving plasma—the filtered product—on the collection plate. Next, 0.5 μL TG assay reagent (Express Biotech International Inc., Frederick, Md.) was deposited on a capture plate (a planar plastic plate, made of PMMA with 1 mm thick and 3 inch by 1 inch wide) and then transferred onto the plasma on the collection plate. The capture plate was hand-pressed against the collection plate, forming a QMAX device, to incubate the TG assay for 1 min. The assay image was then read by an reader adapted iPhone, which was pre-configured to capture and analyze images from QMAX devices.

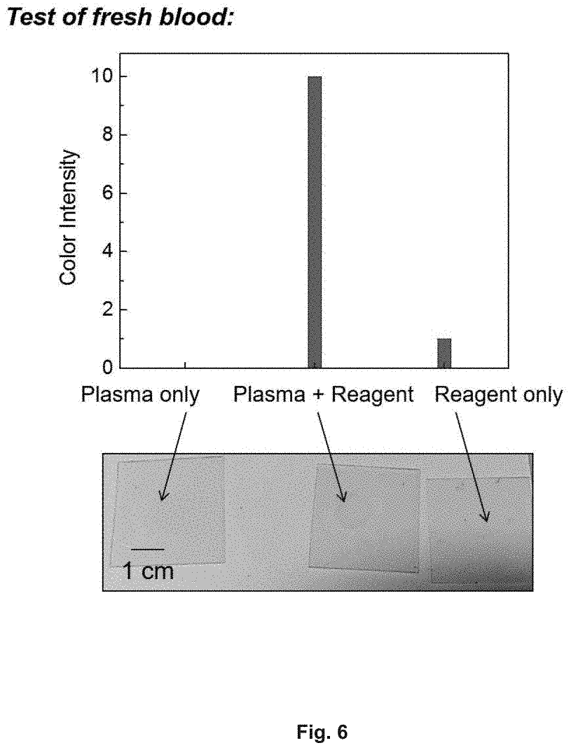

FIG. 6 shows the results of a triglyceride (TG) assay using the filtered products from the experimental filtering device as the assay sample and the QMAX device as the assay device. The bottom panel shows the picture of the QMAX devices used for TG assay and imaging. As shown, a long planar glass plate was used to contact and pressed against all three collection plates that were tested, forming three separate QMAX devices. The TG assay here is a colorimetric assay, in that the assay solution changes color (turns pink) when detecting TG and a higher color intensity indicates a higher level of TG in the assay sample. The top panel shows a bar chart of the color intensity results under three different experimental conditions. The color intensity was close to zero when there was plasma (filtered product) only (left bar), and at a very low level when there was reagent only (right bar). However, the color intensity reached the highest level when the plasma and reagent were both present (center bar), indicating the existence of TGs in the plasma.

The example illustrates again the validity of the devices and methods provided by the present invention. It also clearly demonstrates the ease of combining the present invention with a QMAX process, which can significantly accelerate the sampling/sensing/assaying/processing of the sample and expand the applicability of QMAX devices.

A device of any prior device embodiment, has the function of following tests:

Blood cell tests can include, for example, white blood cell count (WBC or leukocyte count), WBC differential count, red blood cell count or erythrocyte count, hematocrit (Hct), hemoglobin (Hbg), mean corpuscular volume (MCV), mean corpuscular hemoglobin (MCH), mean corpuscular hemoglobin concentration (MCHC), red cell distribution width (RDW), platelet count, and mean platelet volume (MPV).

Blood tests can include, for example, blood glucose test, calcium blood test, cardiac enzyme test, cholesterol and lipid tests, C-reactive protein test, D-dimer test, erythrocyte sedimentation rate (ESR) test, folate test, HbA1C test, HCG test, international normalized ratio (INR) test, iron studies, kidney function tests, liver function tests, magnesium blood test, oestrogen blood test, PSA test, testosterone blood test, thyroid function test, Vitamin B12 test, and Vitamin D test;

Blood tests can include, for example, Rast test to determine the substances a subject is allergic to, ESR test checks for inflammation where red blood cells aggregate, Vitamin B12 test to measure the amount of vitamin B12 (cobalamin) in the blood, HDL test for level of “good cholesterol” in blood, LDL test for level of “bad cholesterol” in the blood, CRP to test for the level of inflammation with the body, CBC to provide 15 different blood test readings; INR is a blood clotting test, LFT (Liver function test) test for the levels of waste products, enzymes and proteins that are processed by the liver, Urea and Electrolytes test to measure the function of kidney, Comprehensive metabolic panel (CMP) provides the overall picture of the metabolism and chemical balance of the body;

Liver function tests can include, for example, T-BIL, D-BIL, TP, ALB, GLO, A/G ratio, ALP, AST, ALT, GGT, and LDH.

Renal function tests can include, for example, Urea, CRE, EGFR, Na, K, and Cl.

Uric acid tests can include, for example, UA.

Hepatitis B tests can include, for example, HBsAg, and Anti-HBs.

Tumors Markers tests can include, for example, CEA, CA15-3, CA125, PSA, and CA19-9.

Thyroid function tests can include, for example, TSH, and F-T4.

Tissue inflammation screening can include, for example, CRP, RA factor, pepsinogen, and ESR.

Sexually transmitted disease screening can include, for example, syphilis TP-Ab, and HIV.

Blood grouping tests can include, for example, ABO, and Rh(D).

Urinalysis can include, for example, appearance, PRO, GLU, BIL, URO, RBC, ET, NIT, LEU, SG, pH, and Urine sediments.

Fecal occult blood tests can include, for example, FOBT.

Smear screening can include, for example, Pap Smear.

Allergies and Sensitivities tests can include, for example, IgE test and IgG test.

Biochemical tests can include, for example, the Kastle-Meyer test for the presence of blood in any biofluid, salicylate testing which is a category of drug, the phadebas test for the presence of saliva for forensic purposes, iodine solution tests for starch, the Van Slyke determination test for specific amino acids, the Zimmermann test for ketosteroids, Seliwanoffs test for differentiating between aldose and ketose sugars, test for lipids, Sakaguchi test for the presence of arginine in protein, Hopkins Cole reaction for the presence of tryptophan in proteins, Nitroprusside reaction for the presence of free thiol groups of cysteine in proteins, Sullivan reaction for the presence of cysteine and cystine in proteins, Acree-Rosenheim reaction for the presence of tryptophan in proteins, Pauly reaction for presence of tyrosine or histidine in proteins, Heller's test for presence of albumin in urine, Gmelin's test for the presence of bile pigments in urine, Hay's test for the presence of bile pigments in urine, and like test.

Biochemical tests can include, for example, Barfoed's test for reducing polysaccharides or disaccharides, Benedict's reagent tests for reducing sugars or aldehydes, Fehling's solution tests for reducing sugars or aldehydes, Molisch's test for carbohydrates, Nylander's test for reducing sugars, Rapid furfural test to distinguish between glucose and fructose, the Bicinchoninic acid assay tests for proteins, Biuret reagent tests for proteins and polypeptides, Bradford protein assay measures protein quantitative, The Phadebas Amylase Test determines alpha-amylase activity, Bial's test to test for pentoses, Urea breath test used to identify infections by Helicobacter pylori, and the Wassermann test which is an antibody test for syphilis.

Organic tests can include, for example, the Carbylamine reaction tests for primary amines, the Griess test for organic nitrite compounds, the Iodoform reaction test for the presence of methyl ketones, or compounds which can be oxidized to methyl ketones, the Schiff test detects aldehydes, Tollens' reagent (Silver Mirror) tests for aldehydes, the Zeisel determination test for the presence of esters or ethers, Lucas' reagent is used to determine mainly between primary, secondary and tertiary alcohols, the Bromine test is used to test for the presence of unsaturation and phenols, radiocarbon dating method to determine the age of an object containing organic material, Baeyer's test to test for alkaline KMnO4, Liebermann's test for the detection of cholesterol, and phthalein dye test to test for phenol.

Inorganic tests can include, for example, Barium chloride test for sulfates, the Beilstein test for halides qualitatively, Borax bead test for certain metals, the Carius halogen method measures halides quantitatively, chemical test for cyanide tests for the presence of cyanide, CN-Copper sulfate tests for presence of water, flame test for metals, the Gilman test for the presence of a Grignard reagent, the Kjeldahl method quantitatively determines the presence of nitrogen, Nessler's reagent for the presence of ammonia, Ninhydrin test for ammonia or primary amines, Phosphate test for phosphate, the sodium fusion test for the presence of nitrogen, sulfur, and halides in a sample, the Zerewitinoff determination for any acidic hydrogen, the Oddy test for acid, aldehydes, and sulfides, Gunzberg's test for the presence of hydrochloric acid, Kelling's test for the presence of lactic acid, Marsh test for the detection of arsenic.

A device of any prior device embodiment, can have the function of following tests:

Composition analysis as identification of fibres, blend analysis, and others.

Color fastness tests in washing, laundering, bleaching, and like tests.

Wet processing analysis for scouring and bleaching a lab sample, and like tests.

Defect analysis of samples.

General chemical tests can include, for example, carbonization, dissolution, stripping and redyeing, absorbency of textiles, bleaching loss, dry shrinkage, and like tests others;

Parameter tests including density, nitrogen content, foaming propensity, emulsion stability, and like tests.

Water, effluent & sludge analysis including pH, density, conductivity, odor, turbidity, total dissolved solids, total hardness, acidity, total chlorine, and like tests.

Eco parameters tests including free formaldehyde, copper, cobalt, lead, mercury, polyvinyl chloride, APEO/NPEO tests, and like tests.

A device of any prior device embodiment, can have one or more of the following functions and purposes:

1) Determine the interactions of a sample with other known substances;

2) Determine the composition of a sample;

3) Provide standard data for other scientific, medical, and quality assurance functions;

4) Validate suitability for end-use;

5) Provide a basis for technical communication;

6) Provide a technical means of comparison of several options;

7) Provide evidence in legal proceedings;

8) Determine if, or verify that, the requirements of a specification, regulation, or contract are met.

### Examples of Separating a Component

A1. A device for separating a component from a composite liquid sample, comprising:

a collection plate having a plurality of spacers that are fixed on one of its surfaces, and a filter having a sample receiving surface and a sample exit surface,

wherein at least a part of the spacers point against and are in contact with the sample exit surface of the filter, forming microcavities confined by the sample exit surface and part of the spacers; and

wherein the filter is configured to separate a component from a part of the sample that flows through the filter from the sample receiving surface toward the collection plate.

B1. A method of separating a component from a composite liquid sample, comprising the steps of:
(1) providing a collection plate having a plurality of spacers on one of its surfaces, and a filter that has a sample receiving surface and a sample exit surface, wherein at least a part of the spacers point against and are in contact with the sample exit surface of the filter, forming microcavities confined by the sample exit surface and said part of the spacers;
(2) depositing the sample on the sample receiving surface of the filter; and
(3) driving at least a part of the deposited sample with a driving force to flow through the filter toward the collection plate, wherein the filter is configured to separate said component from said part of the deposited sample that flows through the filter from the sample receiving surface toward the collection plate.
   D1. A device for plasma extraction from a blood sample, comprising:

a collection plate having a plurality of spacers that are fixed on one of its surfaces, and a filter having a sample receiving surface and a sample exit surface,

wherein at least a part of the spacers point against and are in contact with the sample exit surface of the filter, forming microcavities confined by the sample exit surface and part of the spacers;

wherein the spacers have a uniform height of from 1 to 50 μm and constant inter-spacer distance of from 7 to 50 μm; and

wherein the filter is configured to separate blood cells from a part of the blood sample that flows through the filter from the sample receiving surface toward the collection plate, and the filter is made of a material selected from a group consisting of: silver, glass fiber, ceramic, cellulose acetate, cellulose esters, nylon, polytetrafluoroethylene polyester, polyurethane, gelatin, agarose, polyvinyl alcohol, polysulfone, polyester sulfone, polyacrilonitrile, polyvinylidiene fluoride, polypropylene, polyethylene, polyvinyl chloride, polycarbonate, any other materials that can form porous structure and any combinations thereof, and has an average pore size of from 0.1 to 5 μm.

E1. A method of plasma extraction from a blood sample, comprising the steps of:
(1) providing a collection plate having a plurality of spacers on one of its surfaces, and a filter having a sample receiving surface and a sample exit surface,
   wherein at least a part of the spacers point against and are in contact with the sample exit surface of the filter, forming microcavities confined by the sample exit surface and said part of the spacers, and
   wherein the spacers have a uniform height of from 1 to 50 μm and a constant inter-spacer distance of from 7 to 50 μm;
(2) depositing the blood sample on the sample receiving surface of the filter; and
(3) driving at least a part of the deposited blood sample with a driving force to flow through the filter toward the collection plate,
   wherein the filter is configured to separate blood cells from part of the deposited blood sample that flows through the filter from the sample receiving surface toward the collection plate, and made of the abovementioned material group, and any other materials that can form porous structure and any combinations thereof, and has an average pore size of from 0.1 to 5 μm.
      F1. A device for plasma separation from a blood sample, comprising:

a collection plate and a press plate, both of which have a plurality of spacers that are fixed on one of its surfaces, and a filter having a sample receiving surface and a sample exit surface,

wherein at least a part of the collection plate spacers point against and are in contact with the sample exit surface of the filter, forming microcavities confined by the sample exit surface and said part of the spacers;

wherein the spacers of the collection plate and the press plate have a uniform height in a range of 1 to 50 μm and a constant inter-spacer distance in the range of 7 to 50 μm, respectively;

wherein the filter is configured to separate blood cells from a part of the blood sample that flows through the filter from the sample receiving surface toward the collection plate, and made of the abovementioned material group, and any other materials that can form porous structure and any combinations thereof, and has an average pore size in the range of 0.1 to 5 μm;

wherein the press plate is relatively movable to the collection plate and the filter into different configurations;

wherein one of the configurations is a depositing configuration, in which the press plate is separated, partially or completely, from the collection plate and the filter, the distance between the collection plate and the press plate is not regulated by their spacers, the filter, or the deposited sample; and

wherein another of the configurations is a filtering configuration, in which: the filter is positioned between the press plate and the collection plate, the distance between the collection plate and the press plate is regulated by their spacers, the filter, and the deposited sample, at least a part of the spacers and an inner surface of the press plate press at least a part of the deposited sample against the filter, providing at least a part of the driving force.

G1. A method of plasma extraction from a blood sample, comprising the steps of:
(1) providing a collection plate and a press plate, both of which have a plurality of spacers on one of its surfaces, and a filter having a sample receiving surface and a sample exit surface,
   wherein at least a part of the collection plate spacers point against and are in contact with the sample exit surface of the filter, forming microcavities confined by the sample exit surface and said part of the spacers, and
   wherein the spacers of the collection plate and the press plate have a uniform height of from 1 to 50 μm and a constant inter-spacer distance of from 7 to 50 μm, respectively;
(2) depositing the blood sample on the sample receiving surface of the filter;
(3) placing a press plate having a plurality of spacers on one of its surfaces, to contact with the deposited blood sample, wherein at least a part of the spacers of the press plate point against the sample receiving surface of the filter and are in contact with the deposited sample; and
(4) after the placing step, compressing the press plate against the filter to reduce the distance between the press plate and the filter, and to force at least a part of the deposited blood sample to flow through the filter toward the collection plate,
   wherein the filter is configured to separate blood cells from part of the deposited blood sample that flows through the filter from the sample receiving surface toward the collection plate, and made from the abovementioned material group, and any other materials that can form porous structure and any combinations thereof, and has an average pore size of from 0.1 to 5 μm.
      A2. The device of embodiment A1, wherein the microcavities provide a capillary force that constitutes at least a part of a driving force for causing at least a part of the sample that is deposited on the sample receiving surface to flow through the filter toward the collection plate.
      A3. The device of embodiment A1 or embodiment A2, further comprising a force source providing a first liquid that is configured to provide at least a part of the driving force, wherein the first liquid has low intermiscibility with the sample.
      A4. The device of any one of the prior embodiments, further comprising a force source providing a pressurized gas that is configured to provide at least a part of the driving force.
      A5. The device of any one of the prior embodiments, further comprising a sponge,

wherein the sponge has a compressed state and an uncompressed sate;

wherein the sponge is movable relative to the collection plate and the filter into different configurations;

wherein one of the configurations is a depositing configuration, in which the sponge is in the uncompressed state and separated, partially or completely, from the collection plate and the filter, the distance between the collection plate and the sponge is not regulated by the spacers, the filter, or the deposited sample; and

wherein another of the configurations is a filtering configuration, in which the filter is positioned between the sponge and the collection plate, the distance between the collection plate and the sponge is regulated by the spacers, the filter, and the deposited sample, and the sponge is being converted from the uncompressed state to the compressed state, during which the sponge is configured to provide at least a part of the driving force.

A6. The device of any one of the prior embodiment, further comprising a press plate having a plurality of spacers on one of its surfaces,

wherein the press plate is relatively movable to the collection plate and the filter into different configurations;
wherein one of the configurations is a depositing configuration, in which the press plate is separated, partially or completely, from the collection plate and the filter, the distance between the collection plate and the press plate is not regulated by their spacers, the filter, or the deposited sample; and
wherein another of the configurations is a filtering configuration, in which the filter is positioned between the press plate and the collection plate, the distance between the collection plate and the press plate is regulated by their spacers, the filter, and the deposited sample, and at least a part of the spacers and an inner surface of the press plate press at least a part of the deposited sample against the filter, providing at least a part of the driving force.
   A7. The device of embodiment A6, wherein the press plate spacers have a uniform height of from 0.5 to 100 μm and a constant inter-spacer distance is of from 5 to 200 μm.
   A8. The device of embodiment A6, wherein the press plate spacers have a uniform height of from 1 to 50 μm and a constant inter-spacer distance is of from 7 to 50 μm.
   B2. The method of embodiment B1, wherein the microcavities provide a capillary force that constitutes at least a part of the driving force in step (3).
   B3. The method of embodiment B1 or B2, wherein step (3) comprises depositing a first liquid to contact the deposited sample, the first liquid having low intermiscibility with the sample and configured to provide at least a part of the driving force.
   B4. The method of any one of prior method embodiments, wherein step (3) comprises applying a pressurized gas against the deposited sample, the pressurized gas being configured to provide at least a part of the driving force.
   B5. The method of any one of prior method embodiments, wherein step (3) comprises: (a) contacting a sponge with the deposited sample; (b) compressing the sponge against the filter to provide at least a part of the driving force.
   B6. The method of any one of prior method embodiments, wherein step (3) comprises: (a) placing a press plate having a plurality of spacers on one of its surfaces, to contact with the deposited sample, wherein at least a part of the spacers of the press plate point against the sample receiving surface of the filter and are in contact with the deposited sample; (b) after the placing step (a), compressing the press plate against the filter to reduce the distance between the press plate and the filter, and to provide at least a part of the driving force.
   B7. The method of embodiment B6, wherein the press plate spacers have a uniform height of from 0.5 to 100 μm and a constant inter-spacer distance from 5 to 200 μm.
   B8. The method of embodiment B6, wherein the press plate spacers have a uniform height of from 1 to 50 μm and a constant inter-spacer distance of from 7 to 50 μm.
   B9. The method of any one of prior method embodiments, wherein the compressing step is performed by human hand.
   C1. The device or method of any one of prior embodiments, wherein the collection plate spacers have a predetermined substantially uniform height and a predetermined substantially constant inter-spacer distance.
   C2. The device or method of embodiment C1, wherein the uniform height is from 0.5 to 100 μm and the constant inter-spacer distance is from 5 to 200 μm.
   C3. The device of or method embodiment C1, wherein the uniform height is from 0.5 to 20 μm and the constant inter-spacer distance is from 7 to 50 μm.
   C4. The device or method of any prior embodiments, wherein the filter is a mechanical filter, a chemical filter, a biological filter, or any combination thereof.
   C5. The device or method of any prior embodiments, wherein the filter is made of a material selected from the abovementioned filter materials group, and any other material that can form porous structure, and any combinations thereof.
   C6. The device or method of any prior embodiments, wherein the filter has an average pore size of 10 nm to 500 μm.
   C7. The device or method of any prior embodiments, wherein the filter has an average pore size of 0.1 to 5 μm.
   D2. The device of embodiment D1, wherein the microcavities provide a capillary force that consists at least a part of a driving force for causing at least a part of a sample that is deposited on the sample receiving surface to flow through the filter toward the collection plate.
   E2. The method of embodiment E1, wherein the microcavities provide a capillary force that consists at least a part of the driving force in step (3).
   E3. The method of embodiment E1 or E2, wherein the depositing step comprises: (a) pricking the skin of a human release a droplet of blood onto the skin; and (b) contacting the droplet of blood with the filter without use of a blood transfer tool.
   G2. The method of embodiment G1, wherein the compressing step is performed by human hand.
   G3. The method of embodiment G1 or G2, wherein the depositing step comprises: (a) pricking the skin of a human release a droplet of blood onto the skin; and (b) contacting the droplet of blood with the filter without use of a blood transfer tool.
   H1. The device or method of any one of prior embodiments, wherein each of the plates has a thickness of less than 200 μm.
   H2. The device or method of any one of prior embodiments, wherein each of the plates has a thickness of less than 100 μm.
   H3. The device or method of any one of prior embodiments, wherein each of the plates has an area of less than 5 cm².
   H4. The device or method of any one of prior embodiments, wherein each of the plates has an area of less than 2 cm².
   H5. The device or method of any one of prior embodiments, wherein at least one of the plates is made from a flexible polymer.
   H6. The device or method of any one of prior embodiments, wherein at least one of the plates is a flexible plate, and the thickness of the flexible plate times the Young's modulus of the flexible plate is in the range of 60 to 75 GPa-μm, and the fourth power of the inter-spacer-distance (ISD) divided by the thickness of the flexible plate (h) and the Young's modulus (E) of the flexible plate, ISD⁴/(hE), is equal to or less than 10\hat{ }6 um³/GPa.
   H7. The device or method of any one of prior embodiments, wherein the spaces are fixed on the inner surface of the second plate.
   H8. The device or method of any one of prior embodiments, wherein the spacers are pillars with a cross sectional shape selected from round, polygonal, circular, square, rectangular, oval, elliptical, or any combination of the same.
   H9. The device or method of any one of prior embodiments, wherein the spacers have a pillar shape and a substantially flat top surface, wherein, for each spacer, the ratio of the lateral dimension of the spacer to its height is at least 1.
   H10. The device or method of any one of prior embodiments, wherein each spacer has the ratio of the lateral dimension of the spacer to its height is at least 1.
   H11. The device or method of any one of prior embodiments, wherein the minimum lateral dimension of spacer is less than or substantially equal to the minimum dimension of an analyte in the sample.
   H12. The device or method of any one of prior embodiments, wherein the spacers have a pillar shape, and the sidewall corners of the spacers have a round shape with a radius of curvature at least 1 μm.
   H13. The device or method of any one of prior embodiments, wherein the spacers have a density of at least 100/mm².
   H14. The device or method of any one of prior embodiments, wherein the spacers have a density of at least 1000/mm².
   H15. The device or method of any one of prior embodiments, wherein the spacers have a filling factor of at least 1%, wherein the filling factor is the ratio of the spacer area in contact with the layer of uniform thickness to the total plate area in contact with the layer of uniform thickness.
   H16. The device or method of any one of prior embodiments, wherein the Young's modulus of the spacers times the filling factor of the spacers is equal or larger than 10 MPa, wherein the filling factor is the ratio of the spacer area in contact with the layer of uniform thickness to the total plate area in contact with the layer of uniform thickness.
   H17. The device or method of any one of prior embodiments, wherein

at least one of the plates is flexible, and

for the flexible plate, the fourth power of the inter-spacer-distance (ISD) divided by the thickness of the flexible plate (h) and the Young's modulus (E) of the flexible plate, ISD⁴/(hE), is equal to or less than 10\hat{ }6 um³/GPa.

H18. The device or method of any one of prior embodiments, wherein the spacers are fixed on a plate by directly embossing the plate or injection molding of the plate.
H19. The device or method of any one of prior embodiments, wherein the materials of the plate and the spacers are independently selected from polystyrene, PMMG, PC, COC, COP, or another plastic.
C. Method and Apparatus for Depositing Samples into Compressed Open Flow Device Principles and Certain Examples

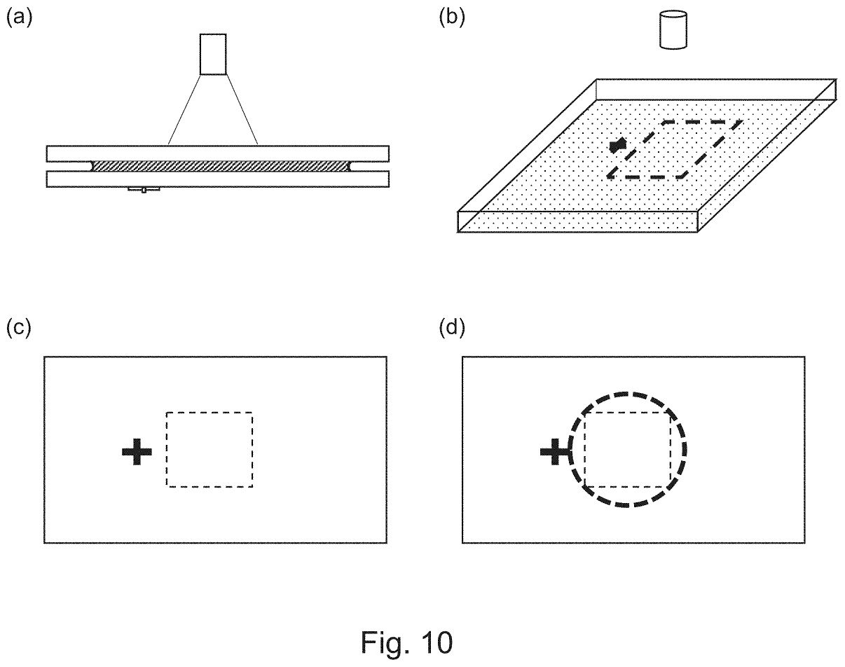

FIG. 9 and FIG. 10 are schematics of a device for sample analysis using compressed open flow (COF) in accordance of some embodiments. The device, as shown FIG. 9 and panel (a) of FIG. 10, includes a first plate and a second plate. Each of the two plates includes an inner surface that has a sample contact area for contacting a sample. The first plate and second plate are movable relative to each other into different configurations. When the device is in the open configuration as shown in FIG. 9, the plates are partially or entirely separated apart, and the average spacing between the sample contact areas of the plates can be, for example, larger than 300 um. In FIG. 9, the liquid sample can be deposited stop the inner surface of the first plate. When the device is in the closed configuration as shown in panel (a) of FIG. 10, the first and second plates have an overlap, and an average spacing between the sample contact areas of the plates is 200 μm or less. In panel (a) of FIG. 10, at least a part of the sample deposited in the open configuration is now placed between the two plates. The liquid sample between the two plates can be imaged by a camera positioned at a distance from the second plate. The combination of the plates and the sample is generally transparent at least at the wavelength of interest.

Panel (b) of FIG. 10 shows a bottom view of the corresponding device in panel (a) of FIG. 10 in accordance with some embodiments. In panel (b) of FIG. 10, the field of view of the camera is shown. A deposition mark (i.e., a recommended drop area for depositing a droplet of a sample; aka.: a compression mark) on the first plate at a location outside the field of view (aka.: sample contact area) of the camera is also shown in panel (b) of FIG. 10. In some embodiments, the deposition mark can have the shape of cross. In other embodiments, the deposition mark can be in the form of other shapes. In some embodiments, the deposition mark can be printed at the outer surface of the first plate. In some embodiments, the deposition mark can be made as a three dimensional (3D) structure at the outer surface of the second plate. For example, the 3D structure at the outer surface of the first plate as shown in panel (a) and panel (b) of FIG. 10 can be formed, for example, during the molding process when the second plate is molding into its designed shape.

In some embodiments, as shown in panel (c) FIG. 10, the first plate can be somewhat translucent or at least partially transparent. The deposition mark at the outer surface of the second plate is visible when viewed through the inner surface of the first plate. During the process of using the device for sample analysis, the liquid sample can be deposited atop the inner surface of the first plate (e.g., as shown in FIG. 9). The deposition mark as shown in panel (c) of FIG. 10 enables the user to deposit the sample at a desired location near the deposition mark. After the sample is deposited at the desired location, the first plate is brought closer to the second plate to change the device from the open configuration to the closed configuration. When the device is changed to the closed configuration, the sample fills the space or cavity between the second plate and the first place. In the closed configuration, a substantially uniform layer of the sample can generally be formed between the second plate and the first plate. Because of the deposition mark, the sample can be deposited at the desired location when the device is in the open configuration; consequently, images of the sample within the field of view of the camera can be captured and stored for further processing, as shown in panel (a) to panel (c) of FIG. 10.

The deposition mark can be designed to have different shape. In one embodiment the deposition mark can be in form of a cross. In one embodiment the deposition mark can be in form of a cross. In another embodiment the deposition mark can be in form of a cross or “plus” symbol that is surrounded by a circle. In another embodiment the deposition mark. can be in form of a circle. In another embodiment the deposition mark can be in form of a star.

Panel (d) of FIG. 10 illustrate that the first plate can also additionally or alternatively be a compression mark. The compression mark is designed to indicate a location or an approximate location for applying a compression force to bring the two plates of the device into the closed configuration. Like the deposition mark, in some embodiments, the compression mark can be printed at the outer surface of the first plate. In some embodiments, the compression mark can be a 3D structure on the outer surface of the first plate. The deposition mark generally has a predetermined position relative to the compression mark. In some embodiments, the compression mark is in a shape that surrounds the imaging area of the sample. The imaging area of the sample generally is the field of view of the camera when the device is set in the position for imaging by the camera. In some embodiments, as shown in panel (d) of FIG. 10, the deposition mark and the compression mark (dotted line circle) are formed by different marks. In some embodiments, the deposition mark and the compression mark can be formed by the same mark. The mark surrounding the imaging area of the sample can be used for both the deposition mark and the compression mark.

### Examples

### Deposition Mark

A1. A device for sample analysis using compressed open flow (COF), comprising:

a first plate, a second plate, and a sample deposition mark, wherein:

the first plate and second plate are movable relative to each other into different configurations, including an open configuration and a closed configuration,

each of the plates comprises an inner surface that has a sample contact area for contacting a sample, and

the first plate has the sample deposition mark that indicates an approximate location for depositing the sample in the open configuration,

wherein in the open configuration the plates are partially or entirely separated apart, and the average spacing between the sample contact areas of the plates is larger than 300 um, and

wherein in the closed configuration the first and second plates have an overlap, at least a part of the sample deposited in the open configuration is between the two plates, and an average spacing between the sample contact areas of the plates can be, for example, 200 μm or less.

A1.1. A device for sample analysis using compressed open flow (COF), comprising:

a first plate, a second plate, and a sample deposition mark, wherein:

the first plate and second plate are movable relative to each other into different configurations, including an open configuration and a closed configuration,

each of the plates comprises an inner surface that has a sample contact area for contacting a liquid sample, and

the first plate has the sample deposition mark that indicates an approximate location for depositing the sample in the open configuration,

wherein the open configuration is a configuration in which the plates are partially or entirely separated apart, and the average spacing between the sample contact areas of the plates is larger than 300 um,

wherein the closed configuration is a configuration in which the first and second plates face each other, at least a part of the sample deposited in the open configuration is confined between the inner surfaces of the two plates, and an average spacing between the sample contact areas of the plates is 200 μm or less, and

wherein the sample deposition mark improves a chance that a substantial portion of the sample is confined between the sample contact areas.

A2. An apparatus for sample analysis using compressed open flow (COF), comprising:

a device of embodiment A1;

a camera that is configured to image at least a part of a sample in the device; and

an adaptor that is configured to position the device and the camera relative to each other, so that the camera images a predetermined viewing location of the device, wherein the predetermined viewing location has a predetermined position relative to the deposition mark of the device.

A3. A method for sample analysis using compressed open flow (COF), comprising the steps of:

having a device of embodiment A1;

depositing, at the open configuration, the sample at or approximately at the location of the sample deposition mark on the first plate, wherein the sample is suspected of containing an analyte;

bringing the device into the closed configuration;

providing a camera;

using an adaptor to position the device and the camera relative to each other, so that the camera images a predetermined viewing location of the device, wherein the predetermined viewing location has a predetermined position relative to the deposition mark of the device.

A4. The device, apparatus, and method of any prior embodiments, wherein the device further comprises, on one of the plates, a compression mark that indicates a location or an approximate location for applying a compression force to bring the two plates into the closed configuration.
A5. The device, apparatus, and method of embodiment A4, wherein the compression mark has a predetermined position relative to the deposition mark.
A6. The device, apparatus, and method of embodiment A4, wherein the compression mark and the deposition mark are a same single mark.

### Compression Mark

B1. A device for sample analysis using compressed open flow (COF), comprising:

a first plate, a second plate, and a compression mark, wherein:

the first plate and second plate are movable relative to each other into different configurations, including an open configuration and a closed configuration,

each of the plates comprises an inner surface that has a sample contact area for contacting a sample, and

one or both plates have the compression mark that indicates an approximate location for application of a compression force to bring the two plates into the closed configuration,

wherein in the open configuration the plates are partially or entirely separated apart, and the average spacing between the sample contact areas of the plates is larger than 300 um, and

wherein in the closed configuration the first and second plates have an overlap, at least a part of the sample deposited in the open configuration is between the two plates, and an average spacing between the sample contact areas of the plates is 200 μm or less.

B1.1. A device for sample analysis using compressed open flow (COF), comprising:

a first plate, a second plate, and a compression mark, wherein:

the first plate and second plate are movable relative to each other into different configurations, including an open configuration and a closed configuration,

each of the plates comprises an inner surface that has a sample contact area for contacting a liquid sample, and

the first plate has the compression mark that indicates an approximate location for applying a compression force to bring the two plates into the closed configuration;

wherein the open configuration is a configuration in which the plates are partially or entirely separated apart, and the average spacing between the sample contact areas of the plates is larger than 300 um,

wherein the closed configuration is a configuration in which the first and second plates face each other, at least a part of the sample deposited in the open configuration is confined between the inner surfaces of the two plates, and an average spacing between the sample contact areas of the plates is 200 μm or less, and

wherein the compression mark improves a uniformity of the sample that is confined between the sample contact areas.

B2. An apparatus for sample analysis using compressed open flow (COF), comprising:

a device of embodiment B1;

a camera that images at least a part of a sample in the device; and

an adaptor that is configured to position the device and the camera relative to each other, so that the camera images a predetermined viewing location of the device, wherein the predetermined viewing location has a predetermined position relative to the compression mark of the device.

B3. A method for sample analysis using compressed open flow (COF), comprising the steps of:

providing a device of embodiment B1;

depositing, at the open configuration, the sample that is suspected of containing an analyte;

bringing the device into the closed configuration by applying a compression force at or approximately at the location of the compression mark;

providing a camera;

using an adaptor to position the device and the camera relative to each other, so that the camera images a predetermined location of the device, wherein the predetermined location has a predetermined position relative to the compression mark of the device.

B4. The device, apparatus, and method of any prior embodiments, further comprising on one of the plates of the device a sample deposition mark that indicates a location or an approximate location for depositing a sample.
B5. The device, apparatus, and method of embodiment B4, wherein the deposition mark has a predetermined position relative to the compression mark.
B6. The device, apparatus, and method of embodiment B4, wherein the compression mark and the deposition mark are a same single mark.

### RELATED DOCUMENTS

The present invention includes a variety of embodiments, which can be combined in multiple ways as long as the various components do not contradict one another. These embodiments include not only aspects of the present invention in the current file, but also aspects of the documents that are herein referenced, incorporated, or to which priority is claimed. The devices, systems, and methods disclosed in the following sections (1)-(9), e.g., definitions, Q-Card, spacer, uniform sample thickness, hinges, opening notches, recessed edge, sliders, smartphone detection system, detection methods, labels, analytes, applications (field and samples), and cloud technologies, including the structure, material, function, variation, dimension and connection thereof, are defined and described in the current application, or in PCT Application (designating U.S.) Nos. PCT/US2016/046437 and PCT/US2016/051775, respectively filed Aug. 10, 2016 and Sep. 14, 2016, and US Provisional Application No. 62/456,065, filed Feb. 7, 2017; 62/426,065, filed Feb. 8, 2017, and 62/456,504, filed Feb. 8, 2017, all of which applications are incorporated in their entireties for all purposes.

### (1) Definitions

The terms “CROF Card (or card)”, “COF Card”, “QMAX-Card”, “Q-Card”, “CROF device”, “COF device”, “QMAX-device”, “CROF plates”, “COF plates”, and “QMAX-plates” are interchangeable, except that in some embodiments, the COF card does not comprise spacers; and the terms refer to a device that comprises a first plate and a second plate that are movable relative to each other into different configurations (including an open configuration and a closed configuration), and that comprises spacers (except some embodiments of the COF card) that regulate the spacing between the plates. The term “X-plate” refers to one of the two plates in a CROF card, wherein the spacers are fixed to this plate. More descriptions of the COF Card, CROF Card, and X-plate are given in the abovementioned provisional application no. 62/456,065.

### (2) Q-Card, Spacer and Uniform Sample Thickness

The devices, systems, and methods herein disclosed can include or use Q-cards, spacers, and uniform sample thickness embodiments for sample detection, analysis, and quantification. In some embodiments, the Q-card comprises spacers, which help to render at least part of the sample into a layer of high uniformity.

### (3) Hinges, Opening Notches, Recessed Edge and Sliders

In some embodiments, the Q-card comprises hinges, notches, recesses, and sliders, which help to facilitate the manipulation of the Q card and the measurement of the samples.

### (4) Q-Card, Sliders, and Smartphone Detection System

In some embodiments, the Q-cards are used together with sliders that allow the card to be read by a smartphone detection system.

### (5) Detection Methods

The devices, systems, and methods herein disclosed can include or be used in various types of detection methods.

### (6) Labels

The devices, systems, and methods herein disclosed can employ various types of labels that are used for analytes detection.

### (7) Analytes

The devices, systems, and methods herein disclosed can be applied to manipulation and detection of various types of analytes (including biomarkers).

### (8) Applications (Field and Samples)

The devices, systems, and methods herein disclosed can be used for various applications (fields and samples).

### (9) Cloud

The devices, systems, and methods herein disclosed can employ cloud technology for data transfer, storage, and/or analysis.

### ADDITIONAL NOTES

Further examples of inventive subject matter according to the present disclosure are described in the following enumerated paragraphs.

As used herein, the terms “adapted” and “configured” mean that the element, component, or other subject matter is designed and/or intended to perform a given function. Thus, the use of the terms “adapted” and “configured” should not be construed to mean that a given element, component, or other subject matter is simply “capable of” performing a given function. Similarly, subject matter that is recited as being configured to perform a particular function may additionally or alternatively be described as being operative to perform that function.

As used herein, the phrase, “for example,” the phrase, “as an example,” and/or simply the terms “example” and “exemplary” when used with reference to one or more components, features, details, structures, embodiments, and/or methods according to the present disclosure, are intended to convey that the described component, feature, detail, structure, embodiment, and/or method is an illustrative, non-exclusive example of components, features, details, structures, embodiments, and/or methods according to the present disclosure. Thus, the described component, feature, detail, structure, embodiment, and/or method is not intended to be limiting, required, or exclusive/exhaustive; and other components, features, details, structures, embodiments, and/or methods, including structurally and/or functionally similar and/or equivalent components, features, details, structures, embodiments, and/or methods, are also within the scope of the present disclosure.

As used herein, the phrases “at least one of” and “one or more of,” in reference to a list of more than one entity, means any one or more of the entity in the list of entity, and is not limited to at least one of each and every entity specifically listed within the list of entity. For example, “at least one of A and B” (or, equivalently, “at least one of A or B,” or, equivalently, “at least one of A and/or B”) may refer to A alone, B alone, or the combination of A and B.

As used herein, the term “and/or” placed between a first entity and a second entity means one of (1) the first entity, (2) the second entity, and (3) the first entity and the second entity. Multiple entity listed with “and/or” should be construed in the same manner, i.e., “one or more” of the entity so conjoined. Other entity may optionally be present other than the entity specifically identified by the “and/or” clause, whether related or unrelated to those entities specifically identified.

Where numerical ranges are mentioned herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, unless otherwise indicated or otherwise evident from the context and understanding of one of ordinary skill in the art.

1. A device for sample analysis facilitated by a separation sheet, comprising:
a first plate; a second plate; a hinge; and a separation sheet, wherein:
(a) the first plate and the second plate are movable relative to each other into a different configuration, the different configuration includes an initial configuration, an open configuration, or a closed configuration, wherein:
(i) each plate comprises an inner surface that has a sample contact area for contacting a sample deposited between the plates,
(ii) at least one of the plates has a thickness of 300 um or less, and
(iii) one of the plates has, in the initial configuration, all its edges, other than the edge connected to the hinge, inside the edges of the other plate;
(b) the hinge is connected to the first plate and the second plate, and the hinge is configured to allow the first plate and the second plate to rotate around the hinge into a different configuration; and
(c) the separation sheet has a thickness of 250 um or less;
wherein:
in the initial configuration, the separation sheet is sandwiched between the two plates and in contact with the two plates; and the separation sheet has an extended portion that is not covered by any of the plates, wherein the extended portion of the separation sheet is configured to facilitate a separation of the two plates;
in the open configuration the first plate and the second plate are partially or entirely separated, and the sample is deposited in the sample contact area on one or both of the plates, and the separation sheet is removed from any contact with one or both of the plates; and
in the closed configuration at least part of the deposited sample is compressed by the two plates into a thin layer.

2. A device for sample analysis facilitated by a separation sheet, comprising:
a first plate; a second plate; a hinge; at least one reagent; and a separation sheet, wherein:
(a) the first plate and the second plate are movable relative to each other into a different configuration, the different configuration includes an initial configuration, an open configuration, or a closed configuration, wherein:
(i) each plate comprises an inner surface that has a sample contact area for contacting a sample deposited between the plates, and
(ii) the second plate has a thickness of 300 um or less;
(b) the hinge is connected to the first plate and the second plate, and the hinge is configured to allow the first plate and the second plate to rotate around the hinge into a different configuration;
(c) the at least one reagent is coated, in the initial configuration, on the sample contact area of at least one of the plates, and
(d) the separation sheet has a thickness of 250 um or less;
wherein:
in the initial configuration, the separation sheet is sandwiched between the two plates and is in contact with the two plates; and the separation sheet is configured to reduce or prevent, in the initial configuration, the at least one regent on one plate to contact the other plate;
in the open configuration the first plate and the second plate are partially or entirely separated, and the sample is deposited in the sample contact area on one or both of the plates, and the separation sheet is removed from any contact with one or both of the plates; and
in the closed configuration at least part of the deposited sample is compressed by the two plates into a sample layer of highly uniform thickness.

3. The device of claim 1, further comprising, in the initial configuration, at least one reagent coated on the sample contact area of at least one of the plates, wherein the separation sheet reduces or prevents, in the initial configuration, the at least one reagent from contact with the other plate.

4. The device of claim 1, further comprising, in the initial configuration, at least one reagent coated on the sample contact area of one of the plates and at least one other reagent in the corresponding location of the sample contact area in the other plate, wherein the separation sheet reduces or prevents, in the initial configuration, the at least one reagent from contact with the at least one other reagent on the corresponding location of the sample contact area of the other plate.

5. The device of claim 1, further comprising spacers attached to inner surface of at least one of the plates and in the sample contact area of one or both of the plates so that in the closed configuration the uniform thickness of the layer is confined by the inner surfaces of the plates and is regulated by the plates and the spacers, and the thickness of the layer is from 0.01 to 200 μm.

6. The device of claim 3, wherein the at least one reagent coated on at least one of the plates is coated on both plates.

7. The device of claim 3, wherein the separation sheet preserves the shelf-life of the at least one reagent, preserves the chemical integrity of the at least one reagent, or both.

8. The device of claim 3, wherein the separation sheet is configured to prevent the at least one reagent on one plate from touching the other plate.

9. The device of claim 1, wherein the separation sheet facilitates the physical separation of the first plate and the second plate from in the initial configuration to an open configuration.

10. The device of claim 1, wherein the separation sheet is constructed of a non-porous material selected from a sheet, fiber sheet, a polymer, a polymer coated paper, or a combination thereof.

11. The device of claim 1, wherein the separation sheet is a material selected from polystyrene, PMMA, PC, COC, COP, or a combination thereof.

12. The device of claim 1, wherein the hinge is configured to substantially maintain a dihedral angle of from 0 to 180 degrees between the first plate and the second plate before and after (0 degrees) an external force is applied to the plates.

13. The device of claim 2, wherein in the initial configuration the second plate has all edges, other than one edge connected to the hinge, inside the corresponding edges of the first plate.

14. The device of claim 2, wherein in the initial configuration the second plate has at least one edge, other than one edge connected to the hinge, inside the corresponding edge of the first plate.

15. The device of claim 1, wherein in the initial configuration the separation sheet has at least one edge that extends over the corresponding edge of the first plate and second plate.

16. The device of claim 1, wherein in the initial configuration the separation sheet has at least one edge that extends over the corresponding edge of the first plate and second plate, and the size of the separation sheet is configured to facilitate opening or separation of the first plate and the second plate from the initial configuration having a dihedral angle of about 0 degrees to the open configuration having a dihedral angle greater than about 0 degrees.

17. A method for using a device for sample analysis facilitated by a separation sheet, comprising:
(i) obtaining the device of claim 1;
(ii) removing the separation sheet from the space between the first plate and the second plate;
(iii) depositing a sample for analysis when the device is in an open configuration;
(iv) after (iii), closing the two plates into a closed configuration, wherein at least part of the sample is deposited in the open configuration; and
(v) applying a compression force by pressing the two plates together to form a layer of highly uniform thickness confined by the inner surfaces of the plates.

18. The method of claim 17, further comprising a step (vi) of analyzing the sample.

19. The method of claim 17, further comprising a step (vi) of analyzing the sample by an enzyme-linked immunosorbent assay (ELISA), radioimmunoassay (MA), immunoblot analysis, immunofluorescent assay (IFA), immunohistochemistry, immunoelectron microscopy (IEM), or immuno-luminescence.

20. The method of claim 17, wherein the sample contains a biomarker selected from a protein, a small molecule, a cell, a particle, a nucleic acid, or a combination thereof.

21. The method of claim 17, further comprising a step (vi) of removing the compression force and where the hinge maintains a dihedral angle between the first plate and the second plate that is from 1 to 30 degrees, including any intermediate values and ranges, from the dihedral angle before the compression force is removed.

22. The device of claim 1, wherein the first plate and the second plate comprise rectangular planar members.

23. The device of claim 1, wherein the first plate and the second plate comprise transparent planar members.

24. The device of claim 1, wherein the first plate and the second plate include a uniform gap or cavity therebetween when in a closed configuration.

25. (canceled)

26. The device of claim 1, wherein the sample contact area comprises a predetermined area configured to make contact with a sample and confine the sample to that predetermined area.

27. The device of claim 1, wherein the separation sheet comprises a flexible planar member including a non-porous material configured to prevent liquid, reagents, debris, or a combination thereof from permeating through the separation sheet.

28. The device of claim 1, wherein the separation sheet is removably attachable to the inner surface of at least the first plate or the second plate, and the separation sheet is configured to protectively enclose the sample contact area.

29. The device of claim 1, wherein the separation sheet comprises an adhesive for removably attaching to at least one of the first plate and the second plate.

30. The device of claim 1, wherein the separation sheet comprises a pressure sensitive or pressure activated adhesive.

31. The device of claim 29, wherein the adhesive comprises an elastomeric compound.

32. The device of claim 29, wherein the adhesive comprises an elastomeric compound selected from the group consisting of acrylics, bio-based acrylate, butyl rubber, ethylene-vinyl acetate (EVA), styrene block copolymers (SBC), or a combination thereof.

33. The device of claim 1, wherein the separation sheet is soluble in water, is soluble in water associated with a sample, or soluble in the sample deposited in the device.

34. The device of claim 1, wherein the separation sheet is soluble in water or is soluble in water of the sample in device when activate by changed temperature, light, or a combination thereof.

35. The device of claim 1, wherein the separation sheet is transparent.

36. The device of claim 1, wherein the separation sheet is non-transparent.

37. The device of claim 1, wherein the separation sheet has a hydrophobic surface.

38. The device of claim 1, wherein the separation sheet has a 430 thickness of 5 um, 10 um, 20 um, 30 um, 50 um, 100 um, 200 um, 300 um, 500 um, including intermediate values and ranges.

39. The device of claim 1, wherein the material of the separation sheet is selected from glass, metal, glass microfiber, cellulose acetate, cotton linter, cellulose, polyethylene, paper, wood fiber, recycled newspaper, vegetable matter, recycled cloth, cloth rags, cellulose fibers from plants, trees, wood pulp, rice, water plants, cotton, or a combination thereof.

40. (canceled)

41. (canceled)

42. A device for assaying a sample, comprising:
a first plate, a second plate, a storage well, a sealing film, and a liquid reagent, wherein:
(a) the first plate and the second plate are movable relative to each other into a different configuration, the different configuration includes an initial configuration, an open configuration, or a closed configuration, wherein:
(i) the first plate and the second plate, respectively, comprise an inner surface that has a sample contact area for contacting a sample deposited between the first plate and the second plate, and
(ii) the first plate having a thickness of 1,000 microns or less,
(b) the storage well having a depth of 250 microns less, and is on the inner surface of the second plate, and
(c) the sealing film is a flexible sheet having a thickness of 150 um or less, wherein, in the initial configuration, the liquid reagent is substantially in the storage well and the sealing film is configured to cover the opening of the storage well to keep the liquid reagent inside the storage well,
wherein:
the initial configuration is a configuration in which both plates are in contact with the sealing film;
the open configuration is a configuration in which the first plate and the second plate are partially or entirely separated, and the sample is deposited on one or both plates; and
the closed configuration is a configuration in which (i) the sealing film is removed from the space between the first plate and the second plate, and (ii) at least part of the sample is compressed by the first plate and the second plate into a layer of highly uniform thickness, and the uniform thickness of the layer confined by the inner surfaces of the plates is in the range of 0.01 to 200 μm.

43. A device for assaying a sample, comprising:
a first plate, a second plate, a storage well, a sealing film, and a liquid reagent, wherein:
(a) the first plate and the second plate are movable relative to each other into a different configuration, the different configuration includes an initial configuration, an open configuration, or a closed configuration, wherein:
(i) the first plate and the second plate, respectively, comprise an inner surface that has a sample contact area for contacting a sample deposited between the first plate and the second plate, and
(ii) the first plate having a thickness of 1,000 microns or less,
(b) the storage well having a depth of 250 microns less, and is on the inner surface of the second plate, and
(c) the sealing film is a flexible sheet having a thickness of 150 um or less, wherein, in the initial configuration, the liquid reagent is substantially in the storage well and the sealing film is configured to seal the opening of the storage well to keep the liquid reagent inside the storage well,
wherein:
the initial configuration is a configuration in which both plates are in contact with the sealing film;
the open configuration is a configuration in which the first plate and the second plate are partially or entirely separated, and the sample is deposited on one or both plates; and
the closed configuration is a configuration in which (i) at least part of the sample is compressed by the first plate and the second plate into a layer of uniform thickness, and the uniform thickness of the layer confined by the inner surfaces of the plates is in the range of 0.01 to 200 μm, and (ii) the sealing film is broken by the spacers releasing the reagent stored in the well to the sample.

44. The device of claim 42, wherein the sealing film is a separation sheet.

45. A method for having a liquid reagent on a device for sample analysis, comprising:
(i) obtaining the device of claim 42;
(ii) removing the sealing film from the space between the first plate and the second plate;
(iii) depositing a sample for analysis when the device is in an open configuration;
(iv) after step (iii), closing the first plate and the second plate into a closed configuration,
wherein at least part of the sample deposited in the open configuration is compressed by the first plate and the second plate into a layer of highly uniform thickness, the uniform thickness of the layer confined by the inner surfaces of the first plate and the second plate is from 0.01 to 200 μm, and the liquid reagent contacts the sample.

46. A device for assaying a sample, comprising:
a flexible first plate having spacers;
a second plate having a storage well, a liquid reagent in the storage well, and a sealing film to seal the liquid reagent in the storage well,
wherein the spacers provide spacing between the first plate and a second plate to form a sample layer having a uniform thickness when the plates are configured into a closed configuration after a sample is deposited on the inner surface of either of the plates, and the spacers puncture the sealing film to release the liquid reagent from the storage well to contact the sample when the plates are compressed together.

47. (canceled)

48. (canceled)

49. (canceled)

50. (canceled)

51. (canceled)

52. A device for assaying a composite sample, comprising:
a first collection plate having spacers attached to one surface of the collection plate and having a reagent coated on the surface of the collection plate having the spacers; and
a filter member situated atop the spacers of the first collection plate, the filter member having an optical structure on the filter member surface contacting the spacers,
wherein:
the filter member receives a liquid containing the composite sample;
a compression force applied to the composite sample separates components in the composite sample into the filter member and the first collection plate;
the reagent coat, if present, contacts separated components in the first collection plate to form a product between the reagent and an analyte; and
the optical structure enhances the detection of the product between the reagent and an analyte.

53. The device of claim 52, further comprising an optical structure in place of the reagent coat on the first collection plate.

54. The device of claim 52, wherein the optical structure is selected from an optical plate, an optical coat, or a combination thereof.

55. The device of claim 52, wherein the compression force is provided by gravity, centrifugation, a human hand, a hydraulic press, a source of compressed air, or a combination thereof.

56. A device for sample analysis facilitated by a mark, comprising:
a first plate, a second plate, and a deposition mark, wherein:
the first plate and second plate are movable relative to each other into different configurations, including an open configuration and a closed configuration,
each of the plates comprises an inner surface that has a sample contact area for contacting a sample to be analyzed, and
either or both of the plates has the deposition mark that indicates an approximate location on the plate for depositing the sample,
wherein in the open configuration the plates are partially or entirely separated apart, and the average spacing between the sample contact areas of the plates is larger than 300 um;
wherein in the closed configuration the first and second plates are compressed together to sandwich the sample into a thin layer of a thickness of 200 μm or less; and
wherein the depostion mark is configured to facilitate the analysis of the sample when the plates are in the closed configuration.

57. A device for sample analysis facilitated by a mark, comprising:
a first plate, a second plate, and a compression mark, wherein:
the first plate and second plate are movable relative to each other into different configurations, including an open configuration and a closed configuration,
each of the plates comprises an inner surface that has a sample contact area for contacting a sample, and
either or both of the plates has the compression mark that indicates an approximate location for iniating a compression of the plates into a closed configuration,
wherein in the open configuration the plates are partially or entirely separated apart, and the average spacing between the sample contact areas of the plates is larger than 300 um, and
wherein in the closed configuration the first and second plates are compressed together to sandwich the sample into a thin layer of a thickness of 200 um or less; and
wherein the compression mark is configured to facilitate the analysis of the sample when the plates in the closed configuration.

58. A device for sample analysis facilitated by a mark, comprising:
a first plate, a second plate, and a filling mark, wherein:
the first plate and second plate are movable relative to each other into different configurations, including an open configuration and a closed configuration;
each of the plates comprises an inner surface that has a sample contact area for contacting a sample; and
either or both of the plates has the filling mark that indicates an approximate area that the sample must fill when the plates are in the closed configuration;
wherein in the open configuration the plates are partially or entirely separated apart, and the average spacing between the sample contact areas of the plates is larger than 300 um;
wherein in the closed configuration the first and second plates are compressed together to sandwich the sample into a thin layer of a thickness of 200 um or less; and
wherein the filling mark is configured to facilitate the analysis of the sample when the plates in the closed configuration.

59. A method for assaying a composite sample, comprising:
(i) obtaining the device of claim 52,
(ii) depositing a sample for analysis on the filter member; and
(iii) providing a compression force to the sample on the filter member to separate the composite sample into the filter member and the first collection plate,
wherein the reagent coat, if present, contacts separated components in the first collection plate to form a product between the reagent and an analyte; and
the optical structure, if present, enhances the detection of the product between the reagent and an analyte.

60. A method for sample analysis facilitated by a deposition mark, comprising:
(i) obtaining the device of claim 56, wherein:
the first plate and second plate are movable relative to each other into different configurations, including an open configuration and a closed configuration,
each of the plates comprises an inner surface that has a sample contact area for contacting a sample to be analyzed, and
either or both of the plates has the deposition mark that indicates an approximate location on the plate for depositing the sample;
(ii) depositing the sample on the deposition mark; and
(iii) compressing the plates,
wherein in the open configuration the plates are partially or entirely separated apart, and the average spacing between the sample contact areas of the plates is larger than 300 um;
wherein in the closed configuration the first and second plates are compressed together to sandwich the sample into a thin layer of a thickness of 200 μm or less; and
wherein the depostion mark is configured to facilitate the analysis of the sample when the plates are in the closed configuration.

61. A method for sample analysis facilitated by a compression mark, comprising:
(i) obtaining the device of claim 57, wherein:
the first plate and second plate are movable relative to each other into different configurations, including an open configuration and a closed configuration,
each of the plates comprises an inner surface that has a sample contact area for contacting a sample to be analyzed, and
either or both of the plates has the compression mark that indicates an approximate location on the plate for compression of the sample;
(ii) depositing the sample on the sample contact area; and
(iii) compressing the plates on the compression mark,
wherein in the open configuration the plates are partially or entirely separated apart, and the average spacing between the sample contact areas of the plates is larger than 300 um;
wherein in the closed configuration the first and second plates are compressed together to sandwich the sample into a thin layer of a thickness of 200 μm or less; and
wherein the compression mark is configured to facilitate the analysis of the sample when the plates are in the closed configuration.

62. A method for sample analysis facilitated by a filling mark, comprising:
obtaining the device of claim 58, wherein:
the first plate and second plate are movable relative to each other into different configurations, including an open configuration and a closed configuration, each of the plates comprises an inner surface that has a sample contact area for contacting a sample to be analyzed, and
either or both of the plates has the filling mark that indicates an approximate location and amount on the plate for depositing the sample;
depositing the sample on the sample contact area to approximate the filling mark; and
compressing the plates,
wherein in the open configuration the plates are partially or entirely separated apart, and the average spacing between the sample contact areas of the plates is larger than 300 um;
wherein in the closed configuration the first and second plates are compressed together to sandwich the sample into a thin layer of a thickness of 200 μm or less; and
wherein the filling mark is configured to facilitate the analysis of the sample when the plates are in the closed configuration.

63. The device of claim 56, wherein the deposition mark comprises a shape of a cross, star, a small circle, or a combination thereof.

64. The device of claim 58, wherein the filling mark comprises a shape of a circle, a square, a triangle, a polygon, or a combination thereof.

65. The device of claim 2, wherein the at least one reagent coated on at least one of the plates is coated on both plates.

66. The device of claim 2, further comprising spacers attached to inner surface of at least one of the plates and in the sample contact area of one or both of the plates so that in the closed configuration the uniform thickness of the layer is confined by the inner surfaces of the plates and is regulated by the plates and the spacers, and the thickness of the layer is from 0.01 to 200 μm.

67. The device of claim 2, wherein the at least one reagent coated on at least one of the plates is coated on both plates.

68. The device of claim 2, wherein the separation sheet preserves the shelf-life of the at least one reagent, preserves the chemical integrity of the at least one reagent, or both.

69. The device of claim 2, wherein the separation sheet is configured to prevent the at least one reagent on one plate from touching the other plate.

70. The device of claim 2, wherein the separation sheet facilitates the physical separation of the first plate and the second plate from in the initial configuration to an open configuration.

71. The device of claim 2, wherein the separation sheet is constructed of a non-porous material selected from a sheet, fiber sheet, a polymer, a polymer coated paper, or a combination thereof.

72. The device of claim 2, wherein the separation sheet is a material selected from polystyrene, PMMA, PC, COC, COP, or a combination thereof.

73. The device of claim 2, wherein the hinge is configured to substantially maintain a dihedral angle of from 0 to 180 degrees between the first plate and the second plate before and after (0 degrees) an external force is applied to the plates.

74. The device of claim 2, wherein in the initial configuration the separation sheet has at least one edge that extends over the corresponding edge of the first plate and second plate.

75. The device of claim 2, wherein in the initial configuration the separation sheet has at least one edge that extends over the corresponding edge of the first plate and second plate, and the size of the separation sheet is configured to facilitate opening or separation of the first plate and the second plate from the initial configuration having a dihedral angle of about 0 degrees to the open configuration having a dihedral angle greater than about 0 degrees.

76. A method for using a device for sample analysis facilitated by a separation sheet, comprising:
(i) obtaining the device of claim 2;
(ii) removing the separation sheet from the space between the first plate and the second plate;
(iii) depositing a sample for analysis when the device is in an open configuration;
(iv) after (iii), closing the two plates into a closed configuration, wherein at least part of the sample is deposited in the open configuration; and
(v) applying a compression force by pressing the two plates together to form a layer of highly uniform thickness confined by the inner surfaces of the plates.

77. The method of claim 76, further comprising a step (vi) of analyzing the sample.

78. The method of claim 76, further comprising a step (vi) of analyzing the sample by an enzyme-linked immunosorbent assay (ELISA), radioimmunoassay (MA), immunoblot analysis, immunofluorescent assay (IFA), immunohistochemistry, immunoelectron microscopy (IEM), or immuno-luminescence.

79. The method of claim 76, wherein the sample contains a biomarker selected from a protein, a small molecule, a cell, a particle, a nucleic acid, or a combination thereof.

80. The method of claim 76, further comprising a step (vi) of removing the compression force and where the hinge maintains a dihedral angle between the first plate and the second plate that is from 1 to 30 degrees, including any intermediate values and ranges, from the dihedral angle before the compression force is removed.

81. The device of claim 2, wherein the first plate and the second plate comprise rectangular planar members.

82. The device of claim 2, wherein the first plate and the second plate comprise transparent planar members.

83. The device of claim 2, wherein the first plate and the second plate include a uniform gap or cavity therebetween when in a closed configuration.

84. The device of claim 2, wherein the sample contact area comprises a predetermined area configured to make contact with a sample and confine the sample to that predetermined area.

85. The device of claim 2, wherein the separation sheet comprises a flexible planar member including a non-porous material configured to prevent liquid, reagents, debris, or a combination thereof from permeating through the separation sheet.

86. The device of claim 2, wherein the separation sheet is removably attachable to the inner surface of at least the first plate or the second plate, and the separation sheet is configured to protectively enclose the sample contact area.

87. The device of claim 2, wherein the separation sheet comprises an adhesive for removably attaching to at least one of the first plate and the second plate.

88. The device of claim 2, wherein the separation sheet comprises a pressure sensitive or pressure activated adhesive.

89. The device of claim 87, wherein the adhesive comprises an elastomeric compound.

90. The device of claim 87, wherein the adhesive comprises an elastomeric compound selected from the group consisting of acrylics, bio-based acrylate, butyl rubber, ethylene-vinyl acetate (EVA), styrene block copolymers (SBC), or a combination thereof.

91. The device of claim 2, wherein the separation sheet is soluble in water, is soluble in water associated with a sample, or soluble in the sample deposited in the device.

92. The device of claim 2, wherein the separation sheet is soluble in water or is soluble in water of the sample in device when activate by changed temperature, light, or a combination thereof.

93. The device of claim 2, wherein the separation sheet is transparent.

94. The device of claim 2, wherein the separation sheet is non-transparent.

95. The device of claim 2, wherein the separation sheet has a hydrophobic surface.

96. The device of claim 2, wherein the separation sheet has a 430 thickness of 5 um, 10 um, 20 um, 30 um, 50 um, 100 um, 200 um, 300 um, 500 um, including intermediate values and ranges.

97. The device of claim 2, wherein the material of the separation sheet is selected from glass, metal, glass microfiber, cellulose acetate, cotton linter, cellulose, polyethylene, paper, wood fiber, recycled newspaper, vegetable matter, recycled cloth, cloth rags, cellulose fibers from plants, trees, wood pulp, rice, water plants, cotton, or a combination thereof.

98. The device of claim 43, wherein the sealing film is a separation sheet.

99. A method for having a liquid reagent on a device for sample analysis, comprising:
(i) obtaining the device of claim 43;
(ii) removing the sealing film from the space between the first plate and the second plate;
(iii) depositing a sample for analysis when the device is in an open configuration;
(iv) after step (iii), closing the first plate and the second plate into a closed configuration,
wherein at least part of the sample deposited in the open configuration is compressed by the first plate and the second plate into a layer of highly uniform thickness, the uniform thickness of the layer confined by the inner surfaces of the first plate and the second plate is from 0.01 to 200 μm, and the liquid reagent contacts the sample.

100. The device of claim 57, wherein the compression mark comprises a shape of a cross, star, a small circle, or a combination thereof.

101. The method of claim 60, wherein the deposition mark comprises a shape of a cross, star, a small circle, or a combination thereof.

102. The method of claim 61, wherein the compression mark comprises a shape of a cross, star, a small circle, or a combination thereof.

103. The method of claim 62, wherein the filling mark comprises a shape of a circle, a square, a triangle, a polygon, or a combination thereof.
